(19) United States
(12) Patent Application Publication
(10) Pub. No.: US 2017/0022487 A1
(43) Pub. Date: Jan. 26, 2017

(54) SYNTHETIC CATALYTIC MIMICS OF ESTERASES, LIPASES OR DESATURASES

(71) Applicants: The Regents of the University of California, Oakland, CA (US); Z-Field Technologies, LLC, West Hollywood, CA (US)

(72) Inventors: Michael J. Heller, Poway, CA (US); Tsukasa Takahashi, San Diego, CA (US); Edward Lewis Sheldon, III, San Diego, CA (US)

(21) Appl. No.: 15/301,604

(22) PCT Filed: Apr. 1, 2015
(86) PCT No.: PCT/US2015/023829

§ 371 Date: Oct. 3, 2016

Related U.S. Application Data
(60) Provisional application No. 61/974,004, filed on Apr. 2, 2014.

(51) Int. Cl. C12N 9/20; C12N 9/16

(52) U.S. Cl. CPC C12N 9/20; C12N 9/16; C12Y 301/00; C12Y 301/01

(57) ABSTRACT

Novel synthetic catalytic structures or “synzymes,” e.g., fatty acid modified polypeptides, with catalytic properties are provided. It is believed that these synthetic catalytic structures mimic some of the precise conformational changes necessary for catalytic activities seen in enzymes. The catalytic properties of these synthetic catalytic structures or synzymes can be further improved by the application of controlled external forces, e.g., electric fields, or fluidized bed.

### CROSS-REFERENCE TO RELATED APPLICATIONS

This application claims priority to and the benefit of U.S. Provisional Application No. 61/974,004, which was filed on Apr. 2, 2014, and is incorporated by reference in its entirety.

### TECHNICAL FIELD

The present invention relates to synthetic catalytic structures (“synzymes”), e.g., fatty acid modified polypeptides, and the methods, devices, and systems that are utilized together with such synthetic catalytic structures.

### BACKGROUND

Of all the macromolecules in living organisms, enzymes represent those which are the most complex in terms of structure and mechanistic properties. Enzymes are able to catalyze the transformation of all other biomolecules, providing the dynamics and very essence of life. Enzymes can aptly be considered natural bio-nanomachines which do chemistry. In particular, enzymes are proteins that accelerate the chemical transformation of a substrate molecule that binds to the active site of the enzyme in a thermodynamically and mechanically favorable manner, resulting in a chemical transformation of the substrate into a product molecule. Such enzyme catalyzed chemical transformations can include hydrolysis, oxidation/reduction, group transfer, isomerization, addition or removal of groups from double bonds, and ligation reactions. Enzymes catalyze reactions with high specificity and enormous rate accelerations, some having turnover numbers of millions of substrate molecules per second.

In the case of certain proteases, a catalytic triad is thought to be primarily responsible for the efficient hydrolysis (cleavage) of amide bonds in proteins and polypeptides, as well as ester bonds in certain biomolecule and synthetic substrates. For a serine protease such as Chymotrypsin, the catalytic triad motif is a close proximity arrangement of the serine (“Ser”) 195, the histidine (“His”) 57 and the aspartate (“Asp”) 102 amino acid residues in the polypeptide chain. In this catalytic triad, the serine hydroxyl group acts as a strong nucleophile, the histidine imidazole group as a general acid/base and the aspartate carboxyl group helps orient the histidine imidazole group and neutralize the charge that develops during the transition states. With the aid of this hydrogen bonding and exchange network within the reaction site, the catalytic triad functions as a reversible charge relay mechanism where protons are thought to be exchanged from one residue to another producing an efficient catalytic mechanism. While the stereochemical fit and binding between the substrate and enzyme is very important, it is the complex three dimensional (“3D”) protein structure which actually produces the dynamic mechanical properties in the catalytic triad that lead to efficient enzyme catalysis and turnover.

Most scientists who study enzymology are well aware that the remarkable catalytic properties of enzymes come from their complex 3D protein structure. Upon binding a substrate molecule, the enzyme carries out a rapid set of precise chemo-mechanical dynamic movements which converts the substrate(s) into the product molecule(s).

Over the past three decades a number of efforts have been made to create synthetic versions of enzymes which are sometimes called synzymes or enzyme mimics. Many of the synzymes are based on peptides, synthetic macromolecules and more recently nanostructures that are designed to closely resemble the active site of an enzyme. While these synthetic structures look similar to the enzyme active site they may not have the unique mechanical or dynamic catalytic properties to transform a substrate molecule into the desired product molecule in a repeated process i.e., turnover. Early work by one of the inventors of the present invention involved synthetic peptide structures which contain the same basic catalytic groups, a cysteine-sulfhydryl/thiol, a histidine-imidazole and an aspartate-carboxyl, that are in found the active site of Papain (Heller M J, Walder J A and Klotz I M, JACS, 99(8): 2780-2785, 1977). The synthetic peptide structures of that study were found not to exhibit any efficient catalytic properties, particularly with regard to turnover.

The natural enzyme Papain is a cysteine protease from the papaya plant, whose active site catalytic triad (Cys 25, His 159, and Asp 158) efficiently catalyzes the hydrolysis (cleavage) of both peptide (amide) bonds and ester bonds. Papain has a catalytic mechanism similar to Chymotypsin; the only difference is that a cysteine sulfhydryl/thiol group is the primary nucleophile in Papain. In Papain catalysis, the cysteine sulfhydryl/thiol group carries out a nucleophilic attack on the substrate amide/ester bond forming an acyl-cysteine intermediate. The histidine imidazole group is involved in the deacylation of the acyl-cysteine intermediate which leads to rapid turnover of the enzyme. In the case of the synthetic peptide structures which mimicked the Papain active site, acyl-group exchange was observed between the acyl-cysteine and the imidazole group however, back-attack by the cysteine sulfhydryl/thiol group prevented catalysis and any turnover in these synthetic peptide mimics. In this particular case, the back-attack is more formally an example of an intra-molecular acyl-transfer reaction between the cysteine sulfhydryl/thiol and the histidine imidazole, where the equilibrium greatly favors the reverse reaction for reforming the acyl-sulfhydryl/thiol group.

Other early work by one of the inventors of the present invention involved using synthetic DNA structures to catalyze the formation of peptide bonds (Walder et al., PNAS USA, 76 (1):51-55, 1979). This work demonstrated the potential for using amino acid modified DNA/RNA hybridizing structures and DNA templates to catalyze amide bond formation for peptide synthesis reactions. While the hybridized DNA/RNA structures provided very close proximity for the reacting groups, very little peptide bond formation was observed in the study.

In more recent work, systems and methods were developed wherein hydroxyl groups and imidazole groups were arranged in small synthetic structures (Roth et al., JACS 127: 325-330, 2005), as well as in nanostructured channels which assured their close proximity (Kisailus et al., PNAS USA, 103(15):5652-5657, 2006). These synthetic structures were designed to mimic the active site of Silicatein, a mineral-synthesizing enzyme that produces filamentous organic/inorganic cores of marine organisms, which utilizes both a serine hydroxyl group and histidine imidazole group for catalysis. Nevertheless, in these studies little or no turnover was observed in either the small synthetic structures or the precision nanostructures. Yet another example involving synthetic synzyme structures is disclosed in U.S. Pat. No. 6,048,690 to Heller et al., which describes the use of an electric field to enhance catalysis in a basic cysteine-histidine peptide immobilized on an electrode surface as a model for heterogeneous catalysis. However, no activity was observed, suggesting the basic peptide structures still require incorporation of other unique properties.

With regard to other enzyme mechanisms and their catalytic groups, some examples include: (1) Enolase, which catalyzes the conversion of 2-phosphoglycerate to phosphoenol-pyruvate uses a lysine amino group and a glutamate carboxyl group along with Mg²⁺ cations in the catalytic process; (2) Lysozyme, which catalyzes the hydrolysis of glycosidic C—O bonds in polysaccharides uses a glutamate carboxyl and an aspartate carboxyl in the catalytic process; (3) DNA polymerase, which catalyzes the synthesis of DNA uses three aspartate carboxyl groups, two Mg²⁺ cations and deoxynucleotide triphosphates (dNTPs) in the catalytic process; (4) Lactate Dehydrogenase, which catalyzes the reduction of pyruvate to lactate uses two arginine quanidinium groups, a histidine imidazole group and the reduced cofactor/coenzyme nicotinamide adenine dinucleotide (NADH) in the catalytic process; and (5) the water splitting/oxygen-evolving complex in plant photosynthesis utilizes tyrosine hydroxyl groups and four Mn²⁺ cations in this unique and highly important catalytic process. Thus, other catalytic groups which include glutamate carboxyl, the lysine amino, the arginine guanidinium and the tyrosine hydroxyl group; as well as metal cations (e.g., Mg, Mn, Ca) and various coenzymes/cofactors/prosthetic groups (e.g., NADH, FAD, ATP, dNTPs, Heme groups) are involved in enzyme catalysis. Such a diversity of catalytic groups is required in order to carry out the catalysis of a variety of other reactions including oxidation and reduction reactions; group transfer reactions; isomerization reactions; reactions involving the addition or removal of groups from double bonds; ligation reactions involving the formation of C—C, C—S, C—O, and C—N bonds by condensation reactions coupled to ATP or other energy rich molecules; and specialized reactions for photosynthetic driven water-splitting, oxygen evolution, and reductions including hydrogen production.

### SUMMARY

The present invention is based in part on the development of novel synthetic catalytic structures or “synzymes”, e.g., fatty acid modified polypeptides comprising a synthetic polypeptide that are from 6 to 30 amino acids total in length attached to a fatty acid; cyclic polypeptides comprising at least proline residues, four glutamic residues, and two histidine residues; or DNA hairpin or origami structures covalently coupled to synthetic peptides comprising glutamic and histidine residues, all with catalytic properties. The fatty acid modified polypeptides comprise a fatty acid attached to either N-terminus or C-terminus of a synthetic peptide. The fatty acid can be selected from the group consisting of palmitic acid, octanoic acid, hexanoic acid, docosahexaenoic acid, lauric acid, nonanoic acid, valeric acid, decanoic acid, oleic acid, arachidic acid, myristic acid, arachidonic acid, linoleic acid, stearic acid, decosanoic acid, tetracosanoic acid, sapienic acid, elaidic acid, vaccenic acid, eicosapentaenoic acid, and erucic acid. The fatty acid modified polypeptides can form micelles with one or more detergents selected from the group consisting of polyoxyethylene octyl phenyl ether (Triton X-100), polyethylene glycol tert-octylphenyl ether (Triton X-114), polysorbate 20 (Tween-20), polysorbate 80 (Tween-80), nonylphenoxypolyethoxylethanol (NP-40), and octylphenoxypolyethoxyethanol (IGEPAL CA-630). Incorporation of the fatty acid modified polypeptides into micelles can enhance reaction rates by providing a local hydrophobic environment within a surrounding aqueous phase. Many substrates of interest, such as triacylglycerols, are hydrophobic and may be partitioned into the micelle, thus concentrating near the synzyme contained in the micelle. The greater local concentration of substrates can enhance the rate of catalysis based on the law of mass action.

These synthetic catalytic structures are thought to mimic the reaction sites of esterases, lipases, and desaturases and include strategically placed catalytic groups, e.g., one or more of a hydroxyl group, a sulfhydryl/thiol group, an imidazole group, and a carboxyl group; and steric groups, e.g., a benzyl group. The catalytic properties of these synthetic catalytic structures can be further improved by the application of controlled external forces, e.g., electric fields, or fluidized beds. Application of these external forces allows relatively simple synthetic catalytic structures to carry out more efficient dynamic mechanistic movements for efficient catalysis and higher turnover rate.

Disclosed herein are modified polypeptides that comprise synthetic polypeptides attached to fatty acids. The synthetic polypeptides are from 6 to 30 amino acids total in length and can contain one or more strategically placed histidine or histidine analog, cysteine or cysteine analog, serine or serine analog, aspartic acid or aspartic acid analog, alanine or alanine analog, and/or phenylalanine or phenylalanine analog residues. The fatty acids are attached to either N-terminus or C-terminus of the synthetic peptides. The fatty acid can be selected from the group consisting of palmitic acid, octanoic acid, hexanoic acid, docosahexaenoic acid, lauric acid, nonanoic acid, valeric acid, decanoic acid, oleic acid, arachidic acid, myristic acid, arachidonic acid, linoleic acid, stearic acid, decosanoic acid, tetracosanoic acid, sapienic acid, elaidic acid, vaccenic acid, eicosapentaenoic acid, and erucic acid.

In some embodiments, the synthetic polypeptides disclosed herein are from 6 to 30 amino acids total in length and include the amino acid sequence X1-X2-X3-X4-X5 (SEQ ID NO:1). X1, X3, and X5 are independently selected from the group consisting of alanine, an alanine analog, phenylalanine and a phenylalanine analog. In some embodiments, X1, X3, and X5 are independently selected from alanine and phenylalanine. X2 and X4 are independently selected from the group consisting of cysteine, a cysteine analog, serine, a serine analog, histidine, and a histidine analog. In some embodiments, X2 and X4 are independently selected from cysteine, serine, and histidine. When X2 is histidine or a histidine analog, then X4 is cysteine or a cysteine analog, or serine or a serine analog. When X4 is histidine or histidine analog, then X2 is cysteine or a cysteine analog, or serine or a serine analog.

The alanine analog can be selected from the group consisting of β-alanine, dehydroalanine, aminoisobutyric acid, valine and norvaline. The phenylalanine analog can be selected from the group consisting of methylphenylalanine, 1,2,3,4-tetrahydroisoquinoline-3-carboxylic acid, phenylglycine, ethyltyrosine, and methyltyrosine. The cysteine analog can be selected from the group consisting of homocysteine and penicillamine. The serine analog can be selected from the group consisting of methylserine, threonine, 2-amino-3-hydroxy-4-methylpentanoic acid, 3-amino-2-hydroxy-5-methylhexanoic acid, 4-amino-3-hydroxy-6-methylheptanoic acid, and 2-amino-3-hydroxy-3-methylbutanoic acid. The histidine analog can be selected from the group consisting of β-(1,2,3-triazol-4-yl)-DL-alanine, and 1,2,4-triazole-3-alanine.

In some embodiments, SEQ ID NO:1 includes only natural amino acids, e.g., alanine, phenylalanine, cysteine, serine, and histidine. In some embodiments, the synthetic polypeptide can include an amino acid sequence selected from any of SEQ ID NO: 2-37.

In some embodiments, X1, X3, and X5 are alanine or alanine analogs. For example, the synthetic polypeptide can include an amino acid sequence selected from any one of SEQ ID NO: 2, 3, 8, 9, 14, 15, 20, 21, 26, 27, and 28. In some embodiments, X1 and X3 are phenylalanine or phenylalanine analogs. For example, the synthetic polypeptide can include an amino acid sequence selected from any of SEQ ID NO: 4-7, 10-13, 16-19, 22-25, and 29-34.

In some embodiments, the synthetic polypeptides include a negatively charged C-terminal residue, e.g., aspartic acid, glutamic acid, methyl aspartic acid, methyl glutamic acid, 2-aminoadipic acid, 2-aminoheptanedioic acid, or iminodiacetic acid. In some embodiments, the C-terminal residue of the synthetic polypeptides is aspartic acid. In some embodiments, the synthetic polypeptides include an N-terminal residue selected from the group consisting of glycine, lysine, arginine, citrulline, ornithine, and 2-amino-3-guanidinopropionic acid. In some embodiments, the N-terminal residue of the synthetic polypeptides is glycine, lysine or arginine.

In some embodiments, the synthetic polypeptides include a catalytic triad consisting of a cysteine or cysteine analog, a histidine or histidine analog, and an aspartic acid or aspartic acid analog. For example, the synthetic polypeptide can include an amino acid sequence selected from any of SEQ ID NO: 8, 10, 12, 14, 16, 18, 20, 22, 24, 26, 27, 29, 30, or 33. In some embodiments, the synthetic polypeptides can include a catalytic triad consisting of a serine or serine analog, a histidine or histidine analog, and an aspartic acid or aspartic acid analog. For example, the synthetic polypeptide can include an amino acid sequence selected from any of SEQ ID NO: 9, 11, 13, 15, 17, 19, 21, 23, 25, 28, 31, 32, or 34.

The synthetic polypeptides can include 6-30, 7-25, 8-20, or 9-15 amino acids total in length. In some embodiments, the synthetic polypeptides include nine amino acids total in length. For example, the synthetic polypeptide can be an amino acid sequence selected from any of SEQ ID NO: 26-34.

Also disclosed herein are compositions comprising one or more modified polypeptides disclosed herein. The compositions can include one or more detergent, e.g., a detergent selected from the group consisting of polyoxyethylene octyl phenyl ether (Triton X-100), polyethylene glycol tert-octylphenyl ether (Triton X-114), polysorbate 20 (Tween-20), polysorbate 80 (Tween-80), nonylphenoxypolyethoxylethanol (NP-40), and octylphenoxypolyethoxyethanol (IGEPAL CA-630). The fatty acid modified polypeptides and the detergents in the compositions can form micelles. Incorporation of the fatty acid modified polypeptides into micelles can enhance reaction rates by providing a local hydrophobic environment within a surrounding aqueous phase. Many substrates of interest, such as triacylglycerols, are hydrophobic and may be partitioned into the micelle, thus concentrating near the synzyme contained in the micelle. The greater local concentration of substrates can enhance the rate of catalysis based on the law of mass action.

Also provided herein are particles that are coated with the fatty acid modified polypeptides disclosed herein. Those particles can be coated with other amphiphilic polymers. The modified polypeptides are interspersed with the amphiphilic polymers on the surface of the particle. Compositions comprising one or more of the particles disclosed herein are also provided.

Provided herein are also methods of facilitating hydrolysis of lipids. In some embodiments, these methods include contacting the lipid to be hydrolyzed with the modified polypeptides or compositions disclosed herein. In some embodiments, the modified polypeptides are immobilized on the inner surface of channels in a cartridge or a flow-through device. In some embodiments, these methods of facilitating hydrolysis of lipids include contacting the lipid with the particles disclosed herein. In some embodiments, the contacting is carried out by floating the particles described herein in a solution comprising the lipid, e.g., in a fluidized bed. The methods can also include the application of an external electric field to facilitate the hydrolysis of the lipid. The external electrical field can be applied in either one direction or in multiple directions.

Also provided herein are synthetic catalytic structures that are thought to mimic the reaction sites of desaturases. For example, cyclic polypeptides comprising the amino acid sequence of Gly-Glu-Ala-Glu-Ala-Glu-Gly-Pro-Gly-His-Ala-Glu-Ala-His-Gly-Pro (SEQ ID NO: 36) are disclosed. The four Glu residues and two His residues of the cyclic polypeptides can bind to two ferrous atoms. Compositions including the cyclic polypeptides are also provided. Another example is compositions that include a DNA hairpin covalently coupled to four identical peptides comprising the amino acid sequence of Ala-Glu-Ala-His (SEQ ID NO: 37) and the DNA hairpin positions the four peptides in close proximity. The Glu and His residues of the four peptides can bind to two ferrous atoms. Compositions that include a DNA origami structure covalently coupled to peptides that are placed in close proximity and have either Glu or His residue at its free terminus are also provided. The Glu and His residues of the peptides can bind to two ferrous atoms. Disclosed herein are also methods of facilitating desaturation of a lipid. These methods include contacting the lipid with compositions comprising these synthetic catalytic structures that are thought to mimic the reaction sites of desaturases.

Also provided herein are kits comprising the compositions disclosed herein. The kit can also include instructions for use that include instructions for catalytic applications of the modified polypeptides. The kit can also include one or more reaction wells, e.g., electric field cuvettes, to be used with the synzymes. The kit can also include software configured to operate on a computer or processor-driven device or apparatus to control the application of the electric fields.

The present disclosure also includes devices and systems that can be used together with the modified polypeptides disclosed herein, e.g., electric field reaction wells, software configured to operate on a computer or processor-driven device or apparatus to control the application of electric fields.

Also provided herein are arrays of synthetic polypeptides. The array can include at least two modified polypeptides as described herein. In some embodiments, the array can include at least five modified polypeptides. In some embodiments, the array can include at least 15 modified polypeptides. In some embodiments, the array of synzymes is attached to a support or substrate, e.g., glass, silicon, or plastic surface, optionally coated with, for example, a porous membrane such as a hydrogel.

As used herein, the term “synthetic polypeptide” refers to a polypeptide that is chemically synthesized, but does not refer to naturally occurring or recombinant polypeptides. More specifically, the term “synthetic polypeptide” refers to a polypeptide formed, in vitro, by joining amino acids or amino acid analogs in a particular order, using well known techniques of synthetic organic peptide synthesis to form the peptide bonds.

The term “analog” is used herein to refer to an amino acid molecule that structurally resembles a reference amino acid molecule, but has been modified to modify the stereochemistry of the amino acid to the non-natural D-configuration, and/or to replace one or more specific substituents of the reference amino acid molecule with an alternate substituent.

The term “amphiphilic” as used herein means dissolvable in aqueous solvents such as, but not limited to, blood in-vivo, as well as in non-aqueous solvents such as, but not limited to, ethanol, methanol, and/or isopropanol. Accordingly, an “amphiphilic polymer” according to embodiments of the invention are dissolvable in both aqueous and non-aqueous solvents.

### DESCRIPTION OF THE DRAWINGS

FIG. 1 illustrates catalytic micelles that comprise the fatty acid modified polypeptides and detergents and mimic lipase.

FIG. 2 illustrates an exemplary back-attack problem.

FIG. 3 illustrates use of an electrical field to prevent the back-attack problem. In step 1, the thiol reacts with an ester substrate, resulting in acylated sulfur in step 2. In step 3, the acyl group transfers to the imidazole. In step 4, negatively biased electrode pulls the acylated imidazole away from the thiol, which is attracted to the positively biased electrode, to prevent back-transfer to the more reactive sulfur. In step 5, the acyl group is released into the surrounding medium. In step 6, the process starts over again with a free thiol able to attack an ester linkage.

FIG. 4 illustrates electric-field-induced deacylation in catalytic micelles that comprise the fatty acid modified polypeptides and detergents. Synzymes embedded in micelles can also be combined with the use of an alternating electrical field to achieve further rate enhancement. In step 1, the acyl-glycerol substrate is added while the electrodes are not energized and therefore, no electrical field. The sulfur, which has a negative charge, is able to react with the ester bond and acquires the fatty acid as an acyl group. In step 2, the acyl group is transferred to the imidazole group. In step 3, the electrodes are energized to pull the negatively charged sulfur away from the acylated imidazole, thereby preventing back-transfer of the acyl group to the sulfur. In step 4, the fatty acid is released from the imidazole into the surrounding medium.

FIG. 5 illustrates catalytic structures that mimic lipase in a flow-through device. Synthetic enzymes are coated onto or covalently linked to the inner surfaces of the channels in the flow-through device or cartridge with a great amount of surface area provided by the channels. The synzymes can be interspersed with amphiphilic polymers composed hydrophobic linker groups with hydrophilic end groups. Typically the end groups would be hydroxyls or other relatively non-reactive groups. The amphiphilic polymers provide a hydrophobic environment to attract hydrophobic substrates. In addition, the amphiphilic polymers minimize crowding or steric interference between active sites. Such amphiphilic polymers can also be used to passivate the surfaces of the channels to prevent the active sites from sticking to the surfaces. As in FIG. 3, the active sites are composed of cysteine and histidine residues with other amino acid residues between them to facilitate the correct orientation of the thiols and imidazoles. The fluid flow through the cartridge can increase the rate of the reaction by bringing the substrate near the active sites and removing the products, thus preventing the products from participating in back reactions.

FIG. 6 illustrates fluidized bed with synzymes linked to particles. The diagram shows synthetic enzymes immobilized on particles can be used in a fluidized bed format. Here the synzymes are interspersed with amphiphilic polymers bound to the surface. Fluid circulation in the fluidized bed enhances the reaction rate by moving the substrate near the synzymes on the particles. Products are removed through a membrane, which blocks the escape of the particles.

FIG. 7 illustrates another fluidized bed embodiment in which the sulfur and imidazole groups are on different particles. Here, the imidazole-bearing beads can be smaller and more numerous than the sulfur-bearing beads. Otherwise, the more reactive sulfur would be likely to participate in a back attack on the acyl group, thus halting the reaction.

FIG. 8 illustrates the use of an electrical field to facilitate the reaction in a flow-through device. In this embodiment, the imidazole groups are linked to the walls of a channel, potentially in a multi-channel cartridge. In the first step, a substrate with an ester bond is combined with a synthetic peptide containing a cysteine residue. The cysteine residue becomes acylated and releases an alcohol. Next, the solution is pumped into the channel to permit reaction with the imidazole anchored on the walls of the channel. Then, the acyl group transfers from the cysteine residue to the imidazole group. Finally an electrical field is applied to separate the free acid, which is attracted to the positively biased electrodes, and the free thiol peptide, which is attracted to the negatively biased electrodes. Now the thiol-containing peptide is free to react with fresh substrate and a new cycle of the process begins.

FIGS. 9A-9C illustrate synzymes that mimic desaturases. Two DNA/peptide structures with Diiron sites including a DNA hairpin structure, a DNA origami structure and a cyclic peptide with a Diiron site. The family of desaturases can be divided into two groups: (1) soluble enzymes with four glutamic groups and two histidine groups at the active site and (2) membrane-associated enzymes, which probably have four histidine groups at the active site. We have taken the active site of the group of soluble desaturases as our guide for the design of synzymes because X ray crystallographic data is available. Based on the X-ray crystallographic data, we have designed three structures: (1) FIG. 9A shows a DNA hairpin covalently coupled to four identical peptides comprising the amino acid sequence of Ala-Glu-Ala-His (SEQ ID NO: 37) and the DNA hairpin positions the four peptides in close proximity. The Glu and His residues of the four peptides can bind to two ferrous atoms; (2) FIG. 9B shows a DNA origami structure that is covalently coupled to three peptides that coordinate two ferrous atoms; and (3) FIG. 9C shows a cyclic peptide consisting of the amino acid sequence of SEQ ID NO: 36 that coordinates with two ferrous atoms.

### DETAILED DESCRIPTION

The present invention is based in part on the development of novel synthetic catalytic structures or “synzymes”, e.g., fatty acid modified polypeptides comprising a synthetic polypeptide that are from 6 to 30 amino acids total in length attached to a fatty acid; cyclic polypeptides comprising at least four glutamic residues and two histidine residues; or DNA hairpin or origami structures covalently coupled to synthetic peptides comprising glutamic and histidine residues, all with catalytic properties. The fatty acid modified polypeptides comprise a fatty acid attached to either N-terminus or C-terminus of a synthetic peptide. The fatty acid can be selected from the group consisting of palmitic acid, octanoic acid, hexanoic acid, docosahexaenoic acid, lauric acid, nonanoic acid, valeric acid, decanoic acid, oleic acid, arachidic acid, myristic acid, arachidonic acid, linoleic acid, stearic acid, decosanoic acid, tetracosanoic acid, sapienic acid, elaidic acid, vaccenic acid, eicosapentaenoic acid, and erucic acid. The fatty acid modified polypeptides can form micelles with one or more detergents selected from the group consisting of polyoxyethylene octyl phenyl ether (Triton X-100), polyethylene glycol tert-octylphenyl ether (Triton X-114), polysorbate 20 (Tween-20), polysorbate 80 (Tween-80), nonylphenoxypolyethoxylethanol (NP-40), and octylphenoxypolyethoxyethanol (IGEPAL CA-630). Incorporation of the fatty acid modified polypeptides into micelles can enhance reaction rates by providing a local hydrophobic environment within a surrounding aqueous phase. Many substrates of interest, such as triacylglycerols, are hydrophobic and may be partitioned into the micelle, thus concentrating near the synzyme contained in the micelle. The greater local concentration of substrates can enhance the rate of catalysis based on the law of mass action.

These synthetic catalytic structures are thought to mimic the reaction sites of esterases, lipases, and desaturases and include strategically placed catalytic groups, e.g., one or more of a hydroxyl group, a sulfhydryl/thiol group, an imidazole group, and a carboxyl group; and steric groups, e.g., a benzyl group. The catalytic properties of these synthetic catalytic structures can be further improved by the application of controlled external forces, e.g., electric fields, or fluidized beds. Application of these external forces allows relatively simple synthetic catalytic structures to carry out more efficient dynamic mechanistic movements for efficient catalysis and higher turnover rate.

### Synzymes

Disclosed herein are modified polypeptides that comprise synthetic polypeptides attached to fatty acids. The synthetic polypeptides are from 6 to 30 amino acids total in length and can contain one or more strategically placed histidine or histidine analog, cysteine or cysteine analog, serine or serine analog, aspartic acid or aspartic acid analog, alanine or alanine analog, and/or phenylalanine or phenylalanine analog residues. The fatty acids are attached to either N-terminus or C-terminus of the synthetic peptides. The fatty acid can be selected from the group consisting of palmitic acid, octanoic acid, hexanoic acid, docosahexaenoic acid, lauric acid, nonanoic acid, valeric acid, decanoic acid, oleic acid, arachidic acid, myristic acid, arachidonic acid, linoleic acid, stearic acid, decosanoic acid, tetracosanoic acid, sapienic acid, elaidic acid, vaccenic acid, eicosapentaenoic acid, and erucic acid.

In some embodiments, the fatty acid modified polypeptides can form micelles with one or more detergents selected from the group consisting of polyoxyethylene octyl phenyl ether (Triton X-100), polyethylene glycol tert-octylphenyl ether (Triton X-114), polysorbate 20 (Tween-20), polysorbate 80 (Tween-80), nonylphenoxypolyethoxylethanol (NP-40), and octylphenoxypolyethoxyethanol (IGEPAL CA-630). Incorporation of the fatty acid modified polypeptides into micelles can enhance reaction rates by providing a local hydrophobic environment within a surrounding aqueous phase. Many substrates of interest, such as triacylglycerols, are hydrophobic and may be partitioned into the micelle, thus concentrating near the synzyme contained in the micelle. The greater local concentration of substrates can enhance the rate of catalysis based on the law of mass action.

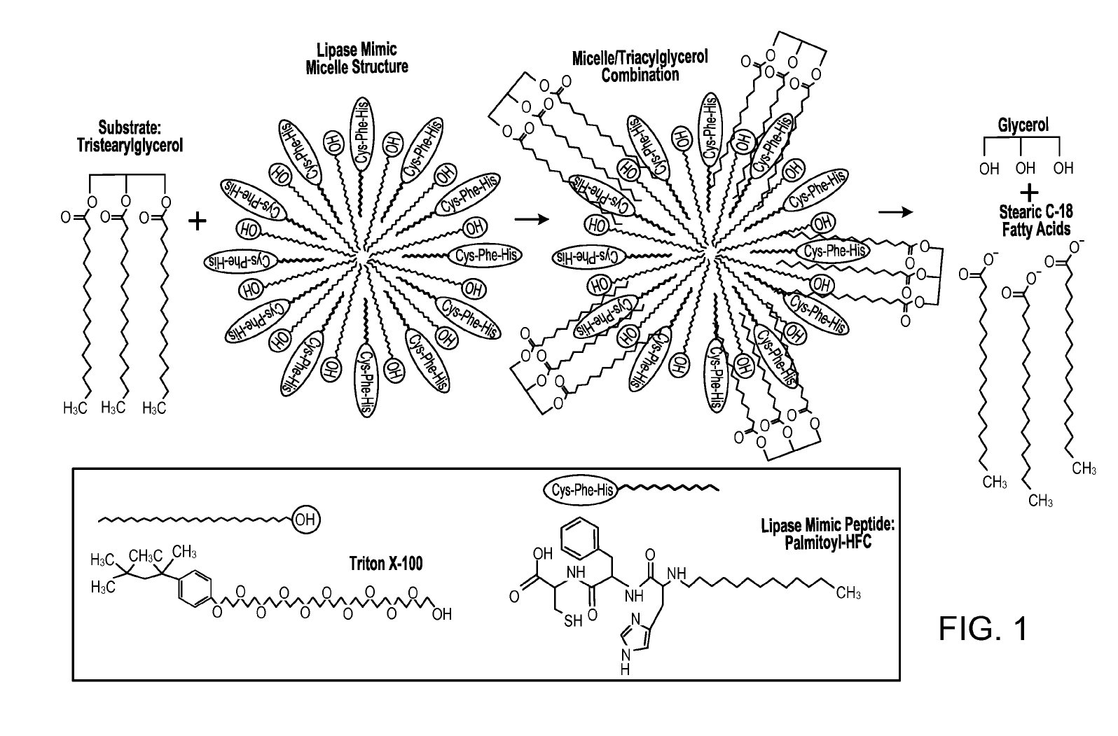

FIG. 1 illustrates catalytic micelles that comprise the fatty acid modified polypeptides and detergents. FIG. 1 shows synzyme constructs that mimic lipase and are composed of synthetic peptides with attached fatty acid hydrophobic tails, embedded in a micelle. The synthetic peptides of the synzyme constructs contain cysteine and histidine residues as well as phenylalanine residues to facilitate the correct orientation of the thiol and imidazole groups. The micelle concentrates the synzymes and the triacylglycerol substrates in the vicinity of each other, which leads to rate acceleration. In addition, the active site of the synzyme, which is the relatively hydrophilic peptide portion, is concentrated at the outer edge of the micelle as is the relatively hydrophilic part of the substrate, which contains the ester linkages. The positioning of the labile ester linkage near the active site of the synzyme also serves to enhance the reaction rate.

The synthetic polypeptides disclosed herein are from 6 to 30 amino acids total in length that can contain one or more strategically placed histidine or histidine analog, cysteine or cysteine analog, serine or serine analog, aspartic acid or aspartic acid analog, alanine or alanine analog, and/or phenylalanine or phenylalanine analog residues. These synthetic polypeptides are believed to utilize one or more of the imidazole group of the histidine or histidine analog, the sulfhydryl/thiol group of the cysteine or cysteine analog, the hydroxyl group of the serine or serine analog, and/or the carboxyl group of aspartic acid or aspartic acid analog, to catalyze hydrolysis of amide or ester bond containing substrates, e.g., without limitation, peptides, proteins, fatty acids, or glycerol esters. Placement of an alanine or alanine analog or phenylalanine or phenylalanine analog between the main catalytic residues, e.g., the histidine or histidine analog and the cysteine or cysteine analog, or the histidine or histidine analog and the serine or serine analog, is thought to modulate proximity of the catalytic groups.

As used herein, the term “synthetic polypeptide” refers to a polypeptide that is chemically synthesized, but does not refer to naturally occurring or recombinant polypeptides. More specifically, the term “synthetic polypeptide” refers to a polypeptide formed, in vitro, by joining amino acids or amino acid analogs in a particular order, using well known techniques of synthetic organic peptide synthesis to form the peptide bonds. For example, polypeptides can be synthesized by solid phase techniques (Roberge et al., Science 269: 202-204, 1995), cleaved from the resin, and purified by preparative high performance liquid chromatography (e.g., Creighton, Proteins Structures And Molecular Principles, WH Freeman and Co, New York, 1983). Automated synthesis can be achieved, for example, using an ABI Peptide Synthesizer (Applied Biosystems) in accordance with the instructions provided by the manufacturer.

The term “analog” is used herein to refer to an amino acid molecule that structurally resembles a reference amino acid molecule, but has been modified to modify the stereochemistry of the amino acid to the non-natural D-configuration, and/or to replace one or more specific substituents of the reference amino acid molecule with an alternate substituent.

The present disclosure also relates to synthetic polypeptides that can include other catalytic groups selected from, but not limited to, the amino group of a lysine or lysine analog, the guanidinium group of an arginine or arginine analog, the carboxyl group of a glutamic acid or glutamic acid analog, and the hydroxyl group of a tyrosine or tyrosine analog.

In some embodiments, the synthetic polypeptides disclosed herein are from 6 to 30 amino acids total in length and include the amino acid sequence X1-X2-X3-X4-X5 (SEQ ID NO:1). X1, X3, and X5 are independently selected from the group consisting of alanine, an alanine analog, phenylalanine and a phenylalanine analog. In some embodiments, X1, X3, and X5 are independently selected from alanine and phenylalanine. X2 and X4 are independently selected from the group consisting of cysteine, a cysteine analog, serine, a serine analog, histidine, and a histidine analog. In some embodiments, X2 and X4 are independently selected from cysteine, serine, and histidine. When X2 is histidine or a histidine analog, then X4 is cysteine or a cysteine analog, or serine or a serine analog. When X4 is histidine or histidine analog, then X2 is cysteine or a cysteine analog, or serine or a serine analog.

The alanine analog can be selected from the group consisting of β-alanine, dehydroalanine, aminoisobutyric acid, valine and norvaline. The phenylalanine analog can be selected from the group consisting of methylphenylalanine, 1,2,3,4-tetrahydroisoquinoline-3-carboxylic acid, phenylglycine, ethyltyrosine, and methyltyrosine. The cysteine analog can be selected from the group consisting of homocysteine and penicillamine. The serine analog can be selected from the group consisting of methylserine, threonine, 2-amino-3-hydroxy-4-methylpentanoic acid, 3-amino-2-hydroxy-5-methylhexanoic acid, 4-amino-3-hydroxy-6-methylheptanoic acid, 2-amino-3-hydroxy-3-methylbutanoic acid. The histidine analog can be selected from the group consisting of β-(1,2,3-triazol-4-yl)-DL-alanine, 1,2,4-triazole-3-alanine.

In some embodiments, SEQ ID NO:1 consists of only natural amino acids, e.g., alanine, phenylalanine, cysteine, serine, and histidine. For example, the synthetic polypeptide can include an amino acid sequence selected from any of SEQ ID NO: 2-37 listed in Table 1. In some embodiments, SEQ ID NO:1 includes one or more amino acid analogs as described above. In some embodiments, SEQ ID NO:1 includes only natural amino acids, but the synthetic polypeptide also include other amino acid analogs.

In some embodiments, when X4 is a histidine or histidine analog, X2 is a cysteine or cysteine analog, the synthetic polypeptides can also include an aspartic acid or aspartic acid analog C-terminal to X5 of the core amino acid sequence. In some embodiments, X4 is histidine, X2 is cysteine, the synthetic polypeptide also includes an aspartic acid C-terminal to X5 of SEQ ID NO:1. For example, the synthetic polypeptide can include an amino acid sequence selected from SEQ ID NO: 8 and 10 listed in Table 1. In some embodiments, the synthetic polypeptide consists of an amino acid sequence of SEQ ID NO: 8 or 10.

In some embodiments, when X4 is a histidine or histidine analog, X2 is a serine or serine analog, the synthetic polypeptide can also include an aspartic acid or aspartic acid analog C-terminal to X5 of the core amino acid sequence. In some embodiments, X4 is histidine, X2 is serine, the synthetic polypeptide also includes an aspartic acid C-terminal to X5 of SEQ ID NO:1. For example, the synthetic polypeptide can include an amino acid sequence of SEQ ID NO: 9 or 11 listed in Table 1. In some embodiments, the synthetic polypeptide consists of an amino acid sequence of SEQ ID NO: 9 or 11.

In some embodiments, when X2 is a histidine or histidine analog, X4 is a cysteine or cysteine analog, the synthetic polypeptides can also include an aspartic acid or aspartic acid analog N-terminal to X1 of SEQ ID NO:1. In some embodiments, X2 is histidine, X4 is cysteine, the synthetic polypeptides also includes an aspartic acid residue N-terminal to X1 of SEQ ID NO: 1. For example, the synthetic polypeptides can include the amino acid sequence of SEQ ID NO: 12. In some embodiments, the synthetic polypeptide consists of the amino acid sequence of SEQ ID NO: 12.

In some embodiments, when X2 is a histidine or histidine analog, X4 is a serine or serine analog, the synthetic polypeptides can also include an aspartic acid or aspartic acid analog N-terminal to X1 of the core amino acid sequence. In some embodiments, X2 is histidine, X4 is serine, the synthetic polypeptides also includes an aspartic acid residue N-terminal to X1 of SEQ ID NO:1. For example, the synthetic polypeptide can include the amino acid sequence of SEQ ID NO: 13. In some embodiments, the synthetic polypeptide consists of the amino acid sequence of SEQ ID NO: 13.

In some embodiments, X1, X3, and X5 are alanine or alanine analogs. The small size of alanine or alanine analogs is thought to bring the catalytic groups of X2 and X4 into close proximity. In some embodiments, X1, X3, and X5 are alanine. For example, the synthetic polypeptide can include an amino acid sequence selected from any one of SEQ ID NO: 2, 3, 8, 9, 14, 15, 20, 21, 26, 27, and 28. In some embodiments, the synthetic polypeptide consists of an amino acid sequence selected from any of SEQ ID NO: 2, 3, 8, 9, 14, 15, 20, 21, and 26-28.

In some embodiments, X1 and X3 are phenylalanine or phenylalanine analogs. The bulky side chain of the phenylalanine or phenylalanine analog residue is thought to slightly bend the polypeptide backbone and thereby move the catalytic groups of X2 and X4 into closer proximity when compared to alanine containing polypeptides. In some embodiments, X1 and X3 are phenylalanine. For example, the synthetic polypeptide can include an amino acid sequence selected from any of SEQ ID NO: 4-7, 10-13, 16-19, 22-25, and 29-34. In some embodiments, the synthetic polypeptide consists of an amino acid sequence selected from any of SEQ ID NO: 4-7, 10-13, 16-19, 22-25, or 29-34.

In some embodiments, the synthetic polypeptides can mimic a lipase or esterase and include a catalytic triad consisting of a cysteine or cysteine analog, a histidine or histidine analog, and/or an aspartic acid or aspartic acid analog. In some embodiments, the synthetic polypeptide includes a catalytic triad consisting of a cysteine, a histidine, and an aspartic acid. For example, the synthetic polypeptide can include an amino acid sequence selected from any of SEQ ID NO: 8, 10, 12, 14, 16, 18, 20, 22, 24, 26, 27, 29, 30, or 33. In some embodiments, the synthetic polypeptide consists of an amino acid sequence selected from any of SEQ ID NO: 8, 10, 12, 14, 16, 18, 20, 22, 24, 26, 27, 29, 30, or 33.

In some embodiments, the synthetic polypeptides can include a catalytic triad consisting of a serine or serine analog, a histidine or histidine analog, and an aspartic acid or aspartic acid analog. In some embodiments, the synthetic polypeptide includes a catalytic triad consisting of a serine, a histidine, and an aspartic acid. For example, the synthetic polypeptide can include an amino acid sequence selected from any of SEQ ID NO: 9, 11, 13, 15, 17, 19, 21, 23, 25, 28, 31, 32, or 34. In some embodiments, the synthetic polypeptide consists of an amino acid sequence selected from any of SEQ ID NO: 9, 11, 13, 15, 17, 19, 21, 23, 25, 28, 31, 32, or 34.

The synthetic polypeptides can include 6-30, 7-25, 8-20, or 9-15 amino acids total in length. In some embodiments, the synthetic polypeptides include nine amino acids total in length. For example, the synthetic polypeptide consists of an amino acid sequence selected from any of SEQ ID NO: 26-34.

In some embodiments, the synthetic polypeptides include a negatively charged C-terminal residue, e.g., aspartic acid, glutamic acid, methyl aspartic acid, methyl glutamic acid, 2-aminoadipic acid, 2-aminoheptanedioic acid, or iminodiacetic acid. In some embodiments, the C-terminal residue of the synthetic polypeptides is aspartic acid. In some embodiments, the synthetic polypeptides include an N-terminal residue selected from the group consisting of glycine, lysine, arginine, citrulline, ornithine, and 2-amino-3-guanidinopropionic acid. In some embodiments, the N-terminal residue of the synthetic polypeptides is glycine, lysine or arginine.

In some embodiments, the synthetic polypeptides can be used in solution for homogenous catalysis applications. For example, these synthetic polypeptides can include an amino acid sequence selected from any of SEQ ID NO: 20-23, 26-29, 31, or 33-34. In some embodiments, the synthetic polypeptide consists of an amino acid sequence selected from any of SEQ ID NO: 20-23, 26-29, 31, or 33-34.

In some embodiments, the synthetic polypeptide can be immobilized or attached onto a solid surface or support, e.g., a location in an electronic device, through a charged group of the synthetic polypeptide. The charged group can be an N-terminal α-amino group, a C-terminal α-carboxyl group, an ε-amino group of lysine or lysine analog, or a sulfhydryl/thiol group of cysteine or cysteine analog. In some embodiments, the charged group is located on a terminal residue of the synthetic polypeptide. In some embodiments, the charged group is located on a residue within one to five amino acids from a terminus of the synthetic polypeptide, and the charged group does not interfere with the catalytic groups. In some embodiments, the charged group is located on a linker conjugated to the synthetic polypeptide. In some embodiments, the synthetic polypeptide is immobilized or attached onto a solid surface or support through the ε-amino group of a terminal lysine residue. For example, the synthetic polypeptide can include the amino acid sequence of SEQ ID NO: 30 or 32.

In some embodiments, the synthetic polypeptides have an overall net negative charge at a neutral pH, which can allow them to be oriented in solution by electrophoretic movement toward the positive electrode when one dimensional direct current electric field is applied. For example, these synthetic polypeptides can have a negatively charged residue, e.g., aspartic acid, glutamic acid, methyl aspartic acid, methyl glutamic acid, 2-aminoadipic acid, 2-aminoheptanedioic acid, or iminodiacetic acid, at one terminus, and an uncharged or weakly positively charged residue at the other terminus. These synthetic polypeptides can include an amino acid sequence selected from any of SEQ ID NO: 26-34. In some embodiments, the synthetic polypeptide consists of an amino acid sequence selected from any of SEQ ID NO: 26-34.

In some embodiments, the N-terminus of the synthetic polypeptides can be protected and uncharged. For example, the N-terminus is protected by, e.g., an acetyl, tert-butyloxycarbonyl, 9-fluorenylmethyloxycarbonyl, benzoyloxycarbonyl, carbobenzyloxy, p-methoxybenzyl, p-methoxybenzyl carbonyl, benzoyl, benzyl, carbamate, p-methoxyphenyl, 3,4-dimethoxybenzyl, or tosyl group. In some embodiments, the N-terminus of the synthetic polypeptides is protected by acetylation. In some embodiments, the C-terminus of the synthetic polypeptides can be protected and uncharged. For example, the C-terminus is protected, e.g., by a methyl, ethyl, benzyl, tert-butyl, silyl, or phenyl group. In some embodiments, both the N-terminus and the C-terminus of the synthetic polypeptides are protected and uncharged.

Exemplary synthetic polypeptide sequences are provided in Table 1:

TABLE 1
Exemplary Synthetic Polypeptide Sequences

| | |
|---|---|
| Ala-Cys-Ala-His-Ala | SEQ ID NO: 2 |
| Ala-Ser-Ala-His-Ala | SEQ ID NO: 3 |
| Phe-Cys-Phe-His-Ala | SEQ ID NO: 4 |
| Phe-Ser-Phe-His-Ala | SEQ ID NO: 5 |
| Phe-His-Phe-Cys-Ala | SEQ ID NO: 6 |
| Phe-His-Phe-Ser-Ala | SEQ ID NO: 7 |
| Ala-Cys-Ala-His-Ala-Asp | SEQ ID NO: 8 |
| Ala-Ser-Ala-His-Ala-Asp | SEQ ID NO: 9 |
| Phe-Cys-Phe-His-Ala-Asp | SEQ ID NO: 10 |
| Phe-Ser-Phe-His-Ala-Asp | SEQ ID NO: 11 |
| Asp-Phe-His-Phe-Cys-Ala | SEQ ID NO: 12 |
| Asp-Phe-His-Phe-Ser-Ala | SEQ ID NO: 13 |
| Ala-Ala-Cys-Ala-His-Ala-Asp | SEQ ID NO: 14 |
| Ala-Ala-Ser-Ala-His-Ala-Asp | SEQ ID NO: 15 |
| Ala-Phe-Cys-Phe-His-Ala-Asp | SEQ ID NO: 16 |
| Ala-Phe-Ser-Phe-His-Ala-Asp | SEQ ID NO: 17 |
| Asp-Phe-His-Phe-Cys-Ala-Gly | SEQ ID NO: 18 |
| Asp-Phe-His-Phe-Ser-Ala-Gly | SEQ ID NO: 19 |
| Gly-Ala-Ala-Cys-Ala-His-Ala-Asp | SEQ ID NO: 20 |
| Gly-Ala-Ala-Ser-Ala-His-Ala-Asp | SEQ ID NO: 21 |
| Gly-Ala-Phe-Cys-Phe-His-Ala-Asp | SEQ ID NO: 22 |
| Gly-Ala-Phe-Ser-Phe-His-Ala-Asp | SEQ ID NO: 23 |
| Asp-Phe-His-Phe-Cys-Ala-Gly-Asp | SEQ ID NO: 24 |
| Asp-Phe-His-Phe-Ser-Ala-Gly-Asp | SEQ ID NO: 25 |
| Gly-Gly-Ala-Ala-Cys-Ala-His-Ala-Asp | SEQ ID NO: 26 |
| Arg-Gly-Ala-Ala-Cys-Ala-His-Ala-Asp | SEQ ID NO: 27 |
| Arg-Gly-Ala-Ala-Ser-Ala-His-Ala-Asp | SEQ ID NO: 28 |
| Arg-Gly-Ala-Phe-Cys-Phe-His-Ala-Asp | SEQ ID NO: 29 |
| Lys-Gly-Ala-Phe-Cys-Phe-His-Ala-Asp | SEQ ID NO: 30 |
| Arg-Gly-Ala-Phe-Ser-Phe-His-Ala-Asp | SEQ ID NO: 31 |
| Lys-Gly-Ala-Phe-Ser-Phe-His-Ala-Asp | SEQ ID NO: 32 |
| Arg-Asp-Phe-His-Phe-Cys-Ala-Gly-Asp | SEQ ID NO: 33 |
| Arg-Asp-Phe-His-Phe-Ser-Ala-Gly-Asp | SEQ ID NO: 34 |
| His-Gly-Gly-Pro-Gly-Gly-His-Gly-Cys-Gly-Asp | SEQ ID NO: 35 |
| Gly-Glu-Ala-Glu-Ala-Glu-Gly-Pro-Gly-His-Ala-Glu-Ala-His-Gly-Pro | SEQ ID NO: 36 |
| Ala-Glu-Ala-His | SEQ ID NO: 37 |

Some prior art peptides that contain catalytic groups such as a serine-hydroxyl or cysteine-sulfhydryl/thiol, a histidine-imidazole, and an aspartate-carboxyl do not exhibit efficient catalytic properties due to ineffective turnover. This is thought to be primarily due to the back-attack problem, where after an acetyl group transfer from a cysteine sulfhydryl/thiol group to a histidine imidazole group occurs, the primary nucleophile (the sulfhydryl/thiol group here) re-attacks the acetyl-imidzole group before it can deacetylate (Heller, et al., JACS; 99(8): 2780, 1977; Kisailus, et al., PNAS, 103(15): 5652-5657, 2006; Carrea, et al., Trends in Biotechnology 23(10):507-1323(10), 2005). As used herein, the term “acylation” (and in some embodiments, “acetylation” if the substrate includes an acetate moiety) refers to the nucleophilic attack (i.e., via the nucleophilic hydroxyl or sulfhydryl/thiol group on the synthetic polypeptide) on the ester or amide bond of the substrate, thus breaking the amide or ester bond and forming the acyl-synthetic polypeptide intermediate structure (i.e., the acylated hydroxyl or sulfhydryl/thiol group) after an amide or ester-containing substrate is contacted with the synthetic polypeptide. As used herein, the term “deacylation” (and in some embodiments, “deacetylation” if the substrate includes an acetate moiety) refer to hydrolyzing the acyl-synthetic polypeptide intermediate and restoring the synthetic polypeptide to its original state (also referred to as “turnover”).

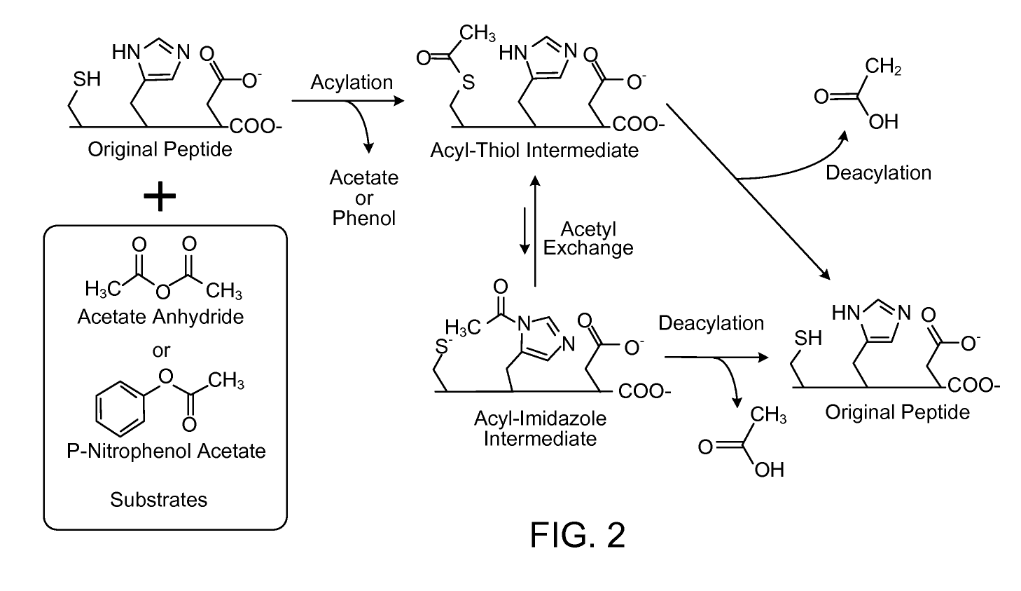

FIG. 2 illustrates the back-attack problem. The synthetic polypeptide in FIG. 2 contains a triad consisting of a cysteine, a histidine, and an aspartic acid residue. The synthetic polypeptide reacts with an ester bond containing substrate such as Acetic Anhydride (AA) or p-Nitrophenol Acetate (pNA). The sulfhydryl/thiol group of the synthetic polypeptide attacks the ester bond in the substrate and forms an acyl-sulfhydryl/thiol intermediate. The product of the cleaved substrate is either a phenol or acetate. The acyl-sulfhydryl/thiol bond is strong, thus deacylation of the acyl-sulfhydryl/thiol group does not usually occur. Instead, the positively charged imidazole group of the histidine residue removes the acyl group from the sulfhydryl/thiol group, and an acyl-imidazole intermediate is formed. Ideally, deacylation of the acyl-imidazole intermediate occurs and restores the synthetic polypeptide to its original state, i.e. turnover of the synthetic polypeptide. However, this can be hindered because of the back attack by the sulfhydryl/thiol group, which causes the acyl group to exchange between the sulfhydryl/thiol and imidazole groups, with the acyl-sulfhydryl/thiol intermediate being more favored than the acyl-imidazole intermediate. Thus this back-attack by the sulfhydryl/thiol group on the acyl-imidazole group prevents the synthetic polypeptide from turning over.

The dynamic movements that can be achieved by the synthetic polypeptides disclosed herein are thought to mimic the mechanistic properties found in real enzymes even without highly complex three dimensional structures. This is achieved by strategically placing the key catalytic groups and steric groups such that the catalytic groups are in close proximity at certain times and under certain conditions, while the proximity can be reduced at other times and under other conditions to eliminate the back-attack problem and facilitate turnover of the synthetic polypeptide. Thus it is within the scope of the present disclosure to provide appropriate steric groups in the structure to produce more favorable proximity of catalytic groups and/or to produce two and three dimensional conformations for inducing improved dynamic mechanistic properties when external forces (e.g. electric fields) are applied.

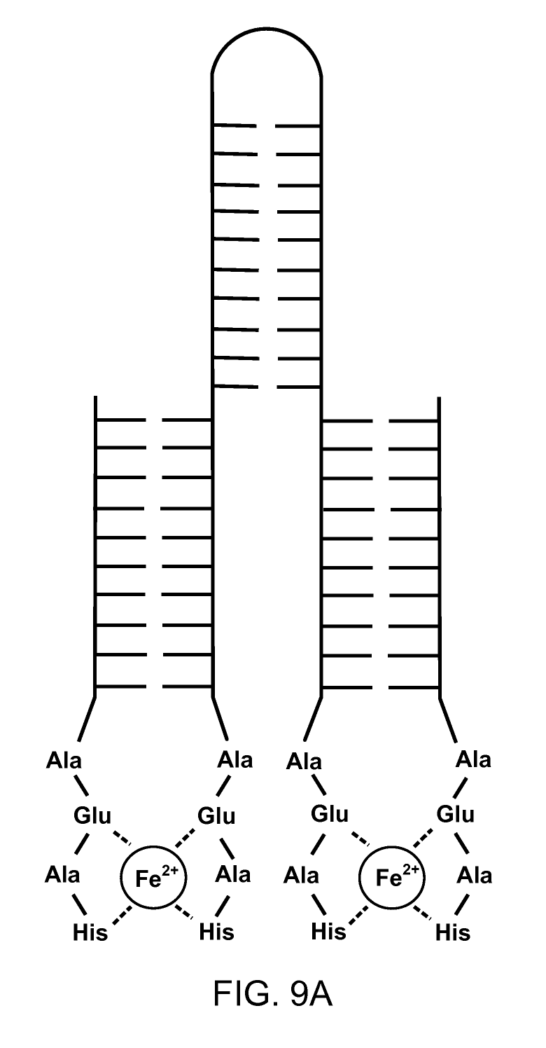

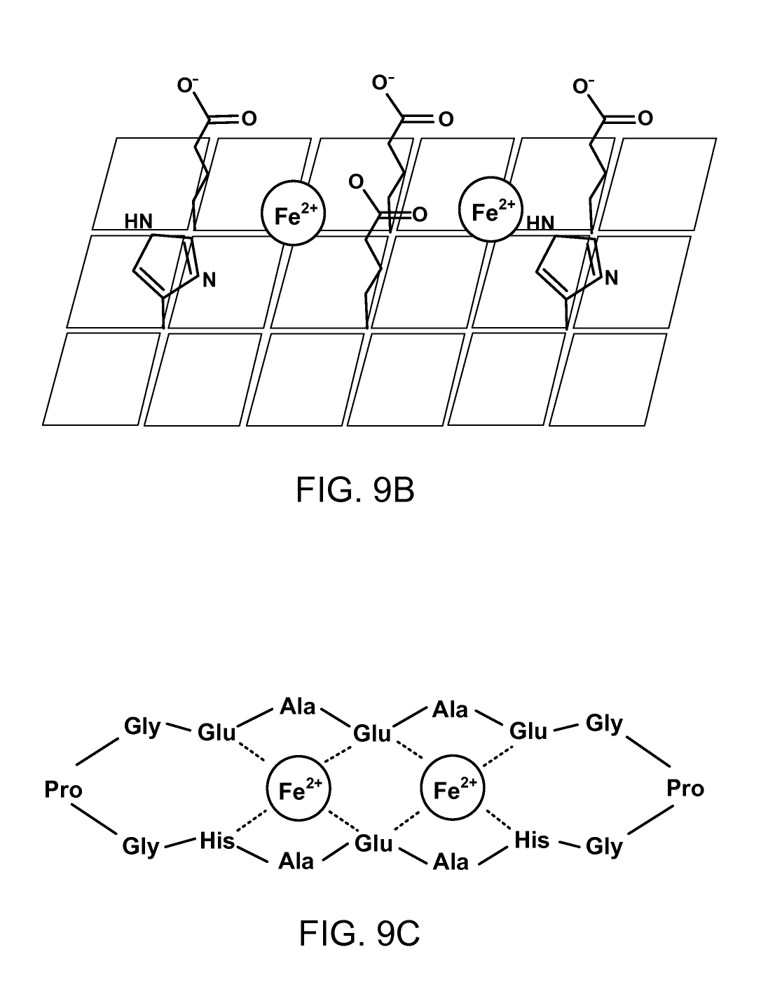

Also provided herein are synthetic catalytic structures that are thought to mimic the reaction sites of desaturases. FIGS. 9A-9C illustrates synzymes that mimic desaturases. Two DNA/peptide structures with Diiron sites including a DNA hairpin structure, a DNA origami structure and a cyclic peptide with a Diiron site. The family of desaturases can be divided into two groups: (1) soluble enzymes with four glutamic groups and two histidine groups at the active site and (2) membrane-associated enzymes, which probably have four histidine groups at the active site. The active site of the group of soluble desaturases were used as guide to design synzymes because X ray crystallographic data is available. Based on the X-ray crystallographic data, we have designed three structures: (1) a DNA hairpin that is covalently coupled to the four peptides that mimic the active site of soluble desaturases and cage two iron atoms in the ferrous state (FIG. 9A), (2) a DNA origami structure that is covalently coupled to three peptides that coordinate two ferrous atoms (FIG. 9B), and (3) a cyclic peptide that coordinates with two ferrous atoms (FIG. 9C).

In some embodiments, the synzymes that mimic desaturases are cyclic peptides that have 16 to 30 amino acid residues and contain four glutamic acid residues and two histidine residues. The four Glu residues and two His residues of the cyclic polypeptides can bind to two ferrous atoms. For example, the cyclic polypeptide can include the amino acid sequence of SEQ ID NO: 36, as illustrated in FIG. 9C. The two proline residues or proline analog in the cyclic polypeptides are believed to create turns in the polypeptide backbone and bring the histidine or histidine analog into close proximity with the glutamic acid or glutamic acid analog, so the glutamic acid and histidine can bind to two iron atoms. Compositions including the cyclic polypeptides are also provided.

In some embodiments, the synzymes that mimic desaturases are compositions illustrated in FIG. 9A, which include a DNA hairpin covalently coupled to four identical peptides comprising the amino acid sequence of Ala-Glu-Ala-His (SEQ ID NO: 37) and the DNA hairpin positions the four peptides in close proximity. The Glu and His residues of the four peptides can bind to two ferrous atoms. In some embodiments, the synzymes that mimic desaturases are compositions illustrated in FIG. 9B, which include a DNA origami structure covalently coupled to peptides that are placed in close proximity and have either Glu or His residue at its free terminus. The Glu and His residues of the peptides can bind to two ferrous atoms.

In addition to synthetic polypeptides, it is also within the scope of the present disclosure to design synzymes using other synthetic molecules, polymers or nanostructures which can provide reactive sulfhydryl/thiol/groups, hydroxyl groups, imidazole groups, carboxyl groups, amino groups or any other useful chemical group. The synthetic catalytic structures can be based on modified DNA (e.g. hairpins or origami) or modified RNA 3D structures. DNA and RNA can also be used to provide hybridization templates to bring catalytic groups, reactants and substrates into close proximity. These synthetic DNA/RNA catalytic structures can be designed such that the application of external forces (e.g., electric fields) can produce efficient catalysis and turnover.

It is also within the scope of the present disclosure to incorporate other groups (charged, polar, apolar) into the synzyme structures which increase the binding affinity of substrate molecules to the catalytic site through charge, hydrogen bonding, hydrophobic binding and van der Waals interactions, i.e., create specific binding sites. All these features are designed to: (1) accelerate the substrate binding event; (2) transform the key catalytic groups into active nucleophiles, electrophiles, general acid/bases for catalyzing hydrolysis of substrates, as well as other reactive groups for catalyzing the oxidation/reduction, isomerization, group transfer; ligation reactions of specific substrate molecules; and hydrogen production; (3) produce a high turnover of the substrate into product, allowing efficient regeneration of the catalyst; and (4) have the synzyme's dynamic catalytic mechanistic properties augmented and enhanced by application of external forces.

Thus the novel synthetic catalytic structures or synzymes disclosed herein include, but are not limited to, synthetic peptides (linear, cyclic, curved/bowed, V-shaped, hairpin), synthetic macromolecules (cyclodextins, synthetic polymers, biopolymers), modified DNA (hairpins, origami structures), modified RNA (3D structures), modified existing proteins, dendrimers, micelles, lipid vesicles, nanoparticles, carbon nanotubes, other nanostructures, microstructures and macrostructures (including but not limited to class, silicon, polymer, plastic and ceramic structures with electrodes), as well as various combinations of these entities and structures. These novel synzyme structures are designed with strategically placed catalytic groups and additional positively or negatively charged groups within the structure; and/or positively or negatively charged entities bound to the synthetic synzyme structure.

### Method of Facilitating Hydrolysis or Desaturation of Lipids Using Synzymes

Provided herein are also methods of facilitating hydrolysis of lipids using the synzymes described herein. In some embodiments, these methods include contacting the lipid to be hydrolyzed with the modified polypeptides or compositions disclosed herein. The contacting step can be performed under such conditions that a cysteine or cysteine analog, or a serine or serine analog, of the synthetic polypeptides can act as a nucleophilic group to attack the ester bond. Under these conditions, the ester bond in the lipid substrate is cleaved and an acyl-synthetic polypeptide intermediate is formed, e.g., an acyl-sulfhydryl/thiol intermediate (when, for example, cysteine is the nucleophilic group) or an acyl-hydroxyl intermediate (when, for example, serine is the nucleophilic group) is formed. The positively charged imidazole group of the histidine or histidine analog removes the acyl group from the sulfhydryl/thiol or hydroxyl group, and an acyl-imidazole intermediate is formed. The physical proximity between the acyl-imidazole group and the sulfhydryl/thiol or hydroxyl group can be modified to prevent back-attack and facilitate deacylation and turnover of the synthetic polypeptides.

The catalytic rate using the synzymes disclosed herein can be further enhanced by using external forces, e.g., electric fields. These external forces, e.g., direct current electric fields, are believed to enable the synthetic polypeptides to carry out the dynamic mechanistic movements necessary for more efficient catalysis and higher turnover. Thus in some embodiments, a method of hydrolyzing a lipid can include a step of contacting the lipid with one or more modified polypeptides described herein; and a step of applying an external force, e.g., an electric field. The contacting step can be performed as described above. The external electric field can be applied to reduce the physical proximity of the acyl-imidazole intermediate and a nucleophilic sulfhydryl/thiol or hydroxyl group of the synthetic polypeptide. The external electrical field can be applied in either one direction or in multiple directions. The application of electrical field can include a single step of applying a directional or an oscillating electric field, or multiple steps of applying directional and oscillating electric fields. For example, when multiple steps of electric field application are utilized, a first directional electric field can be applied for several microseconds to one second to orient the synthetic polypeptide; a second stronger directional electric field can then be applied to position an acyl-sulfhydryl/thiol or acyl-hydroxyl group into close proximity with an imidazole group of the synthetic polypeptide and thereby facilitate formation of an acyl-imidazole intermediate; and then a third oscillating electric field that oscillates at a desired frequency, e.g., from 1 kHz to 1 MHz, can be applied to reduce the physical proximity of the acyl-imidazole intermediate and a nucleophilic sulfhydryl/thiol or hydroxyl group of the synthetic polypeptide. Thus the application of one or more electric fields can be used to facilitate turnover of the synthetic polypeptides.

It is within the scope of the present disclosure to use electric fields and/or other external forces to: (1) produce more active nucleophiles or electrophiles by changing pKa; (2) prevent back-attack in oxidation/reduction and other reactions; (3) orient synthetic synzyme structures for more efficient catalysis for homogeneous (in solution) catalysis; (4) flex and/or open and close synthetic synzyme structures for more efficient catalysis and turnover; (5) concentrate substrate molecules at active site locations; and (6) rapidly remove product molecules from the active site locations.

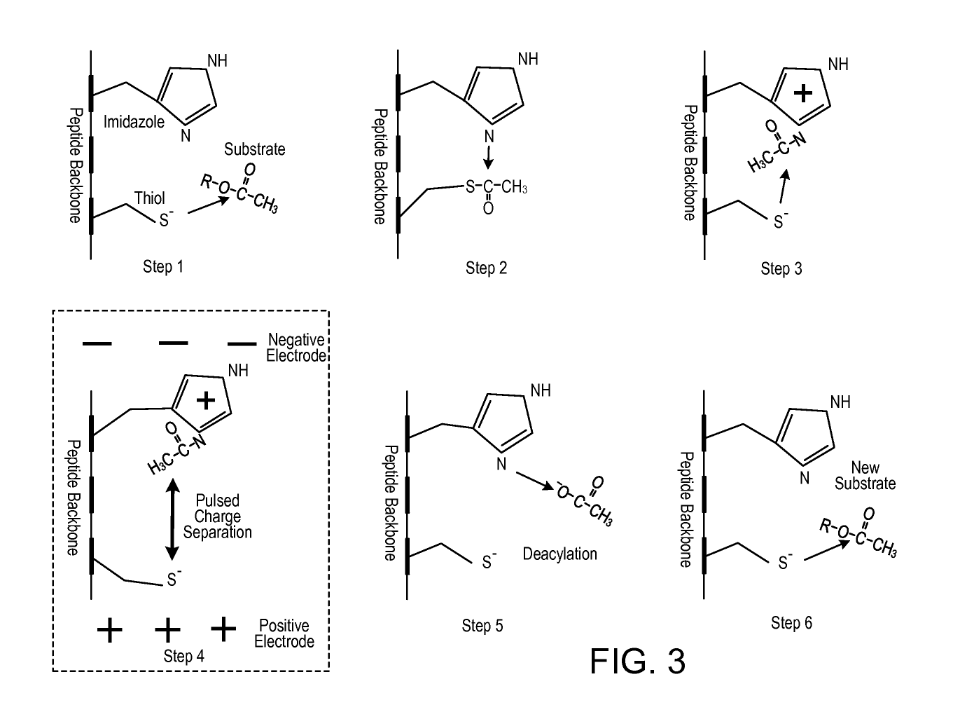

FIG. 3 illustrates use of an electrical field to prevent the back-attack problem. In step 1, the thiol reacts with an ester substrate, resulting in acylated sulfur in step 2. In step 3, the acyl group transfers to the imidazole. In step 4, negatively biased electrode pulls the acylated imidazole away from the thiol, which is attracted to the positively biased electrode, to prevent back-transfer to the more reactive sulfur. In step 5, the acyl group is released into the surrounding medium. In step 6, the process starts over again with a free thiol able to attack an ester linkage.

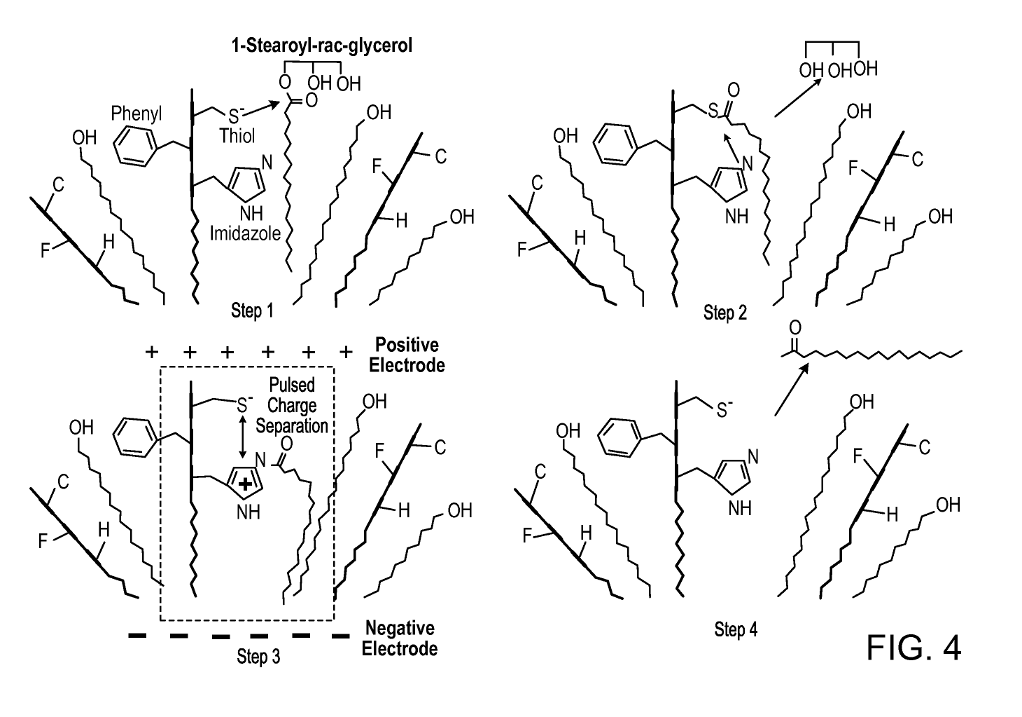

FIG. 4 illustrates electric-field-induced deacylation in catalytic micelles that comprise the fatty acid modified polypeptides and detergents. Synzymes embedded in micelles can also be combined with the use of an alternating electrical field to achieve further rate enhancement. In step 1, the acyl-glycerol substrate is added while the electrodes are not energized and therefore, no electrical field. The sulfur, which has a negative charge, is able to react with the ester bond and acquires the fatty acid as an acyl group. In step 2, the acyl group is transferred to the imidazole group. In step 3, the electrodes are energized to pull the negatively charged sulfur away from the acylated imidazole, thereby preventing back-transfer of the acyl group to the sulfur. In step 4, the fatty acid is released from the imidazole into the surrounding medium.

In some embodiments, the modified polypeptides described herein are immobilized on the inner surface of channels in a cartridge or a flow-through device. In some embodiments, the methods of facilitating hydrolysis of lipids include contacting the lipid with the particles disclosed herein. In some embodiments, the contacting is carried out by floating the particles described herein in a solution comprising the lipid, e.g., in a fluidized bed.

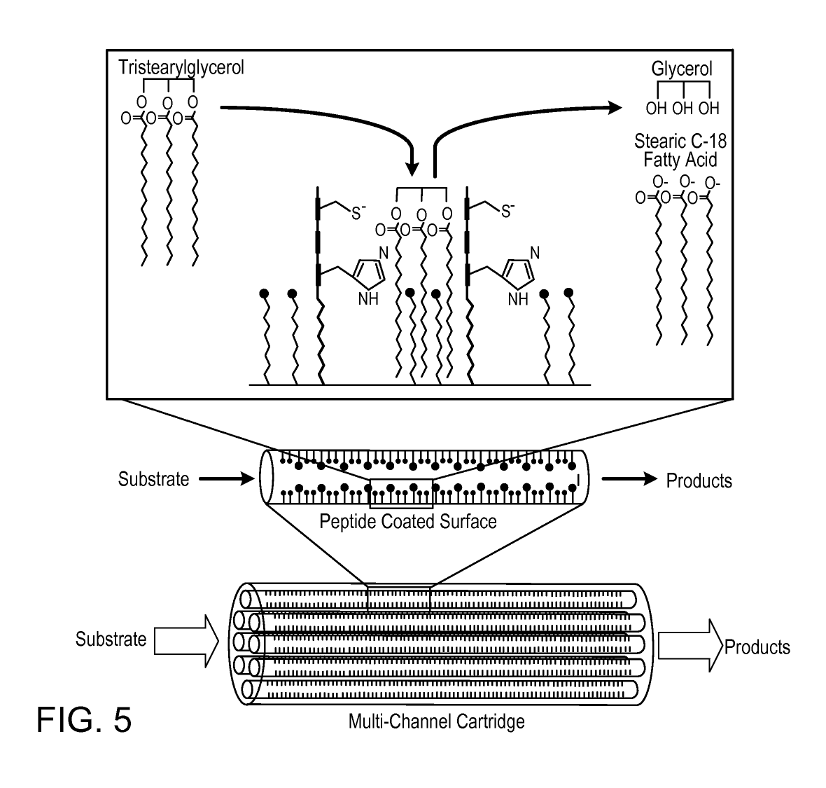

FIG. 5 illustrates catalytic structures that mimic lipase in a flow-through device. Synthetic enzymes are coated onto or covalently linked to the inner surfaces of the channels in the flow-through device or cartridge with a great amount of surface area provided by the channels. The synzymes can be interspersed with amphiphilic polymers composed hydrophobic linker groups with hydrophilic end groups. Typically the end groups would be hydroxyls or other relatively non-reactive groups. The amphiphilic polymers provide a hydrophobic environment to attract hydrophobic substrates. In addition, the amphiphilic polymers minimize crowding or steric interference between active sites. Such amphiphilic polymers can also be used to passivate the surfaces of the channels to prevent the active sites from sticking to the surfaces. As in FIG. 3, the active sites are composed of cysteine and histidine residues with other amino acid residues between them to facilitate the correct orientation of the thiols and imidazoles. The fluid flow through the cartridge can increase the rate of the reaction by bringing the substrate near the active sites and removing the products, thus preventing the products from participating in back reactions.

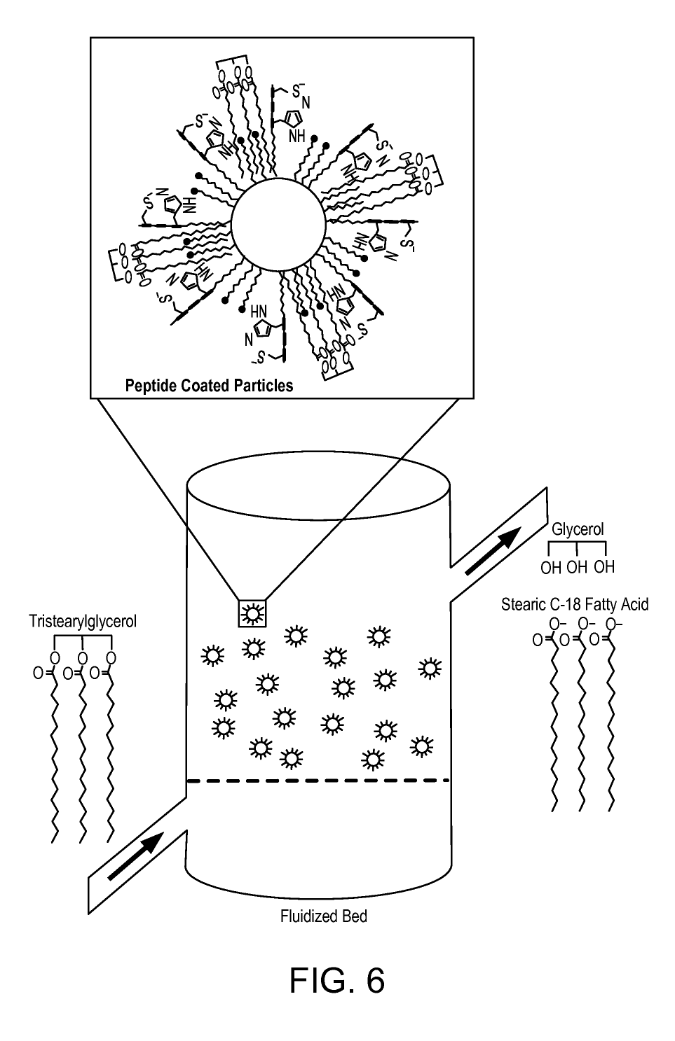

FIG. 6 illustrates fluidized bed with synzymes linked to particles. The diagram shows synthetic enzymes immobilized on particles can be used in a fluidized bed format. Here the synzymes are interspersed with amphiphilic polymers bound to the surface. Fluid circulation in the fluidized bed enhances the reaction rate by moving the substrate near the synzymes on the particles. Products are removed through a membrane, which blocks the escape of the particles.

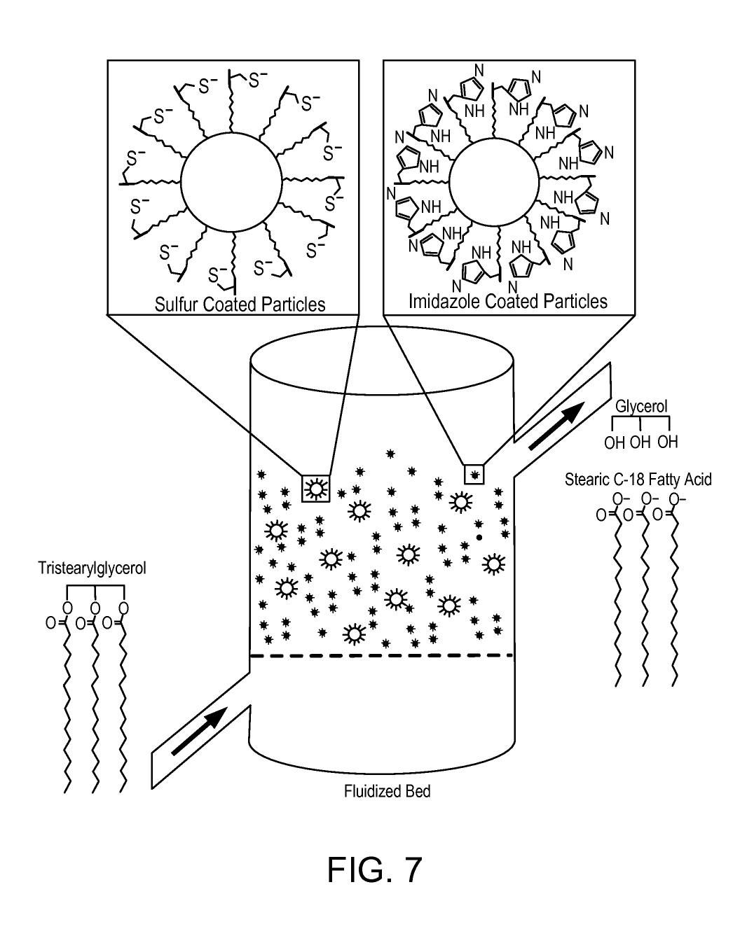

FIG. 7 illustrates another fluidized bed embodiment in which the sulfur and imidazole groups are on different particles. Here, the imidazole-bearing beads can be smaller and more numerous than the sulfur-bearing beads. Otherwise, the more reactive sulfur would be likely to participate in a back attack on the acyl group, thus halting the reaction.

In some embodiments, the amphiphilic polymer is a non-ionic thermoplastic polymer or co-polymer. For example, the amphiphilic polymer or co-copolymer can be hydroxypropyl cellulose (HPC), polyvinyl pyrrolidone (PVP), iodinated HPC, iodinated PVP (povidone iodine). In some embodiments, the amphiphilic polymer is an ionic thermoplastic polymer or co-copolymer. For example, the amphiphilic polymer or co-copolymer can be poly (methyl vinyl ether-alt-maleic acid monobutyl ester) (available under the trade name Gantrez ES-425, from International Specialty Products (ISP), Wayne, N.J.) or poly (methyl vinyl ether-alt-maleic acid monoethyl ester) (available under the trade name Gantrez ES-225, from International Specialty Products (ISP), Wayne, N.J.). In some embodiments, the amphiphilic polymer or co-polymer may not be fully amphiphilic. For example, hydroxypropyl methyl cellulose (HPMC) is not fully soluble in non-aqueous solvent, however some grades are soluble in a solution which contains approximately 10% water and 90% non-aqueous solvent.

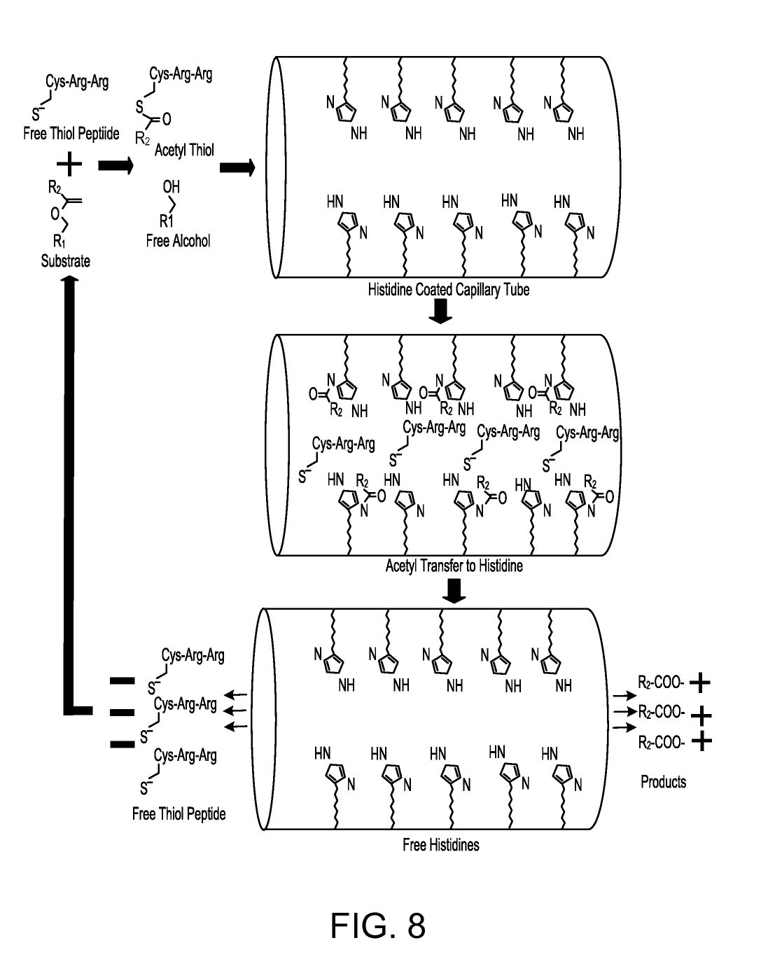

FIG. 8 illustrates the use of an electrical field to facilitate the reaction in a flow-through device. In this embodiment, the imidazole groups are linked to the walls of a channel, potentially in a multi-channel cartridge. In the first step, a substrate with an ester bond is combined with a synthetic peptide containing a cysteine residue. The cysteine residue becomes acylated and releases an alcohol. Next, the solution is pumped into the channel to permit reaction with the imidazole anchored on the walls of the channel. Then, the acyl group transfers from the cysteine residue to the imidazole group. Finally an electrical field is applied to separate the free acid, which is attracted to the positively biased electrodes, and the free thiol peptide, which is attracted to the negatively biased electrodes. Now the thiol-containing peptide is free to react with fresh substrate and a new cycle of the process begins.

Disclosed herein are also methods of facilitating desaturation of a lipid. These methods include contacting the lipid with compositions comprising the synthetic catalytic structures that are thought to mimic the reaction sites of desaturases, e.g., the catalytic structures illustrated in FIGS. 9A-9C.

Devices and Systems Used with the Synzymes

The present disclosure also includes devices and systems that can be used together with the synzymes disclosed herein. These devices and systems can provide controlled application of external forces to synzymes to produce more efficient catalysis. The external forces include but are not limited to electric field, electronic, electrical, electrophoretic, dielectrophoretic (DEP), electrokinetic, electroosmotic, optical, photonic, magnetic, acoustical, fluidic, mechanical, thermal forces as well as various combinations of these external forces. Devices with one, two or three dimensional (2D/3D) arrangements of electrode structures (e.g. Pt, Pd, Au, carbon) that allow for application of direct current (DC) or alternating current (AC) electric fields in continuous and/or pulsed and/or oscillated with polarity reversal modes to be applied to the synzymes in solution or on supports. In the case of using DC and/or AC electric fields for synthetic synzyme structures on supports (heterogeneous catalysis), this would include, but not be limited to, the nano/micro and macroelectrode structures (e.g., Pt, Pd, Au, carbon) on supports (e.g., glass, silicon, plastic) which can be over-layered with porous structures (e.g., hydrogels) to which the synthetic synzyme structures are attached. These devices can have one dimensional (1D), 2D or 3D arrangements of electrodes to: (1) produce DC (>1 volt) electric fields for electrophoretic induced dynamic movements of the synthetic synzyme structures on the support; (2) produce DC (<1 volt) electric fields for producing short range (double-layer) induced dynamic movements of the synthetic synzyme structures when they are attached very close to or directly to the electrodes; and (3) produce AC electric fields for achieving dielectrophoretic (DEP) induced dynamic movements of the synthetic synzyme structures. Associated electronic equipment (e.g., DC power supplies, frequency generators) allows various combinations of AC and/or DC electric fields to be applied in continuous and/or pulsed and/or oscillated with polarity reversal scenarios in three dimensions (3D) around the synthetic synzyme structures in solution (homogeneous catalysis); as well as for synthetic synzyme structures on supports (heterogeneous catalysis).

These devices and systems can be scaled up or down for nano/microscopic applications, intermediate lab-scale applications and for macroscale or industrial, energy (both renewable and non-renewable) and environmental applications; including but not limited to green biomass processing and energy conversions such as cellulose hydrolysis, starch hydrolysis and solar driven water splitting catalysis for hydrogen production. The formats of the devices and systems include but are not limited to various forms of homogeneous (in solution) catalysis, heterogeneous (on support) catalysis which includes fluidized beds as well as various hybrid combinations. Some examples include but are not limited to three dimensional porous support structures with synzymes immobilized within the structures, whose catalytic activity can be enhanced by application of external forces (e.g., electric field), and through which substrates can be flowed into the 3D immobilized synzyme structure and reaction products flowed out of the structure. Such 3D hybrid structures would have the advantages of both homogeneous and heterogeneous catalysis. It is also possible to develop hybrid formats for gas phase catalysis.

In some embodiments, a computer/processor-driven device or apparatus can be configured to design the synthetic polypeptides and other synthetic catalytic structures disclosed herein. For example, a user wishing to design a synzyme having one or more particular characteristics, e.g., a certain rate of turnover, a structure containing one or more particular catalytic groups, enters one or more parameters into the computer/processor-driven device or apparatus, and one or more appropriate synthetic catalytic structures are designed and presented to the user. Such parameters can include, but are not limited to, the particular desired characteristics of the structure. Based on such characteristics, the computer/processor-driven device can utilize, e.g., software using predefined modeling mechanisms or algorithms to determine structures that meet the user's needs. Accordingly, a database or data repository can be utilized to store models, profiles, algorithms, and other data needed to determine the appropriate structure(s). If a user wishes to design synzymes for homogeneous or heterogeneous catalysis applications, the user can specify the type of application in which the synzyme to be designed will be utilized. If the user wishes to design a synthetic synzyme structure with a hand-off mechanism, the user can input such a characteristic as a parameter to be used by the computer/processor-driven device to arrive at an appropriate structure, e.g., one with two histidine groups. Alternatively, a user can enter, e.g., desired catalytic groups, and the computer/processor-driven device can be configured to provide a plurality of possible synzyme structures that have the desired catalytic groups.

It should also be noted that in accordance with another embodiment of the present application, a software application/system/module configured to operate on a computer/processor-driven device or apparatus can be utilized to control the application of external forces to synthetic catalytic structures disclosed herein. For example, such a software application can be used in conjunction with a reaction cell, such as that illustrated in FIGS. 5a and 5b, to program the period of time over which a first directional electric field is applied, the strength of the second directional electric field to be applied, and at what frequency the reverse-polarity electric field is to be oscillated. As also disclosed herein, if a user wishes to apply a continuous or pulsed electric field to a synzyme structure, in which case, the user is given the ability to specify such characteristics of the external force to be applied.

### Arrays and Kits

Also provided herein are arrays of modified polypeptides. The array can include at least two modified polypeptides as described herein. In some embodiments, the array can include at least five modified polypeptides. In some embodiments, the array can include at least 15 modified polypeptides. In some embodiments, the array of synzymes is attached to a support or substrate, e.g., glass, silicon, or plastic surface, optionally coated with, for example, a porous membrane such as a hydrogel.

Also provided herein are kits of modified polypeptides. The kit can include one or more modified polypeptides as described herein. The kit can also include instructions for use and other reagents and devices. Instructions for use can include instructions for catalytic applications of the modified polypeptides. The instructions for use can be in a paper format or on a CD or DVD. The kit can also include one or more reaction wells, e.g., electric field cuvettes. The kit can also include software configured to operate on a computer or processor-driven device or apparatus to control the application of the electric fields.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A modified polypeptide comprising a synthetic polypeptide attached to a fatty acid,
wherein the synthetic polypeptide comprises the amino acid sequence X₁-X₂-X₃-X₄-X₅ (SEQ ID NO:1),
wherein X₁, X₃, and X₅ are independently selected from the group consisting of alanine, an alanine analog, phenylalanine, and a phenylalanine analog; and X₂ and X₄ are independently selected from the group consisting of cysteine, a cysteine analog, serine, a serine analog, histidine, and a histidine analog;
wherein when X₂ is histidine or a histidine analog, X₄ is cysteine or a cysteine analog, or serine or a serine analog;
wherein when X₄ is histidine or a histidine analog, X₂ is cysteine or a cysteine analog, or serine or a serine analog;
wherein the synthetic polypeptide is from 6 to 30 amino acids total in length;
wherein the alanine analog is selected from the group consisting of β-alanine, dehydroalanine, aminoisobutyric acid, valine and norvaline;
wherein the phenylalanine analog is selected from the group consisting of methylphenylalanine, 1,2,3,4-tetrahydroisoquinoline-3-carboxylic acid, phenylglycine, ethyltyrosine, and methyltyrosine;
wherein the cysteine analog is selected from the group consisting of homocysteine and penicillamine;
wherein the serine analog is selected from the group consisting of methylserine, threonine, 2-amino-3-hydroxy-4-methylpentanoic acid, 3-amino-2-hydroxy-5-methylhexanoic acid, 4-amino-3-hydroxy-6-methylheptanoic acid, and 2-amino-3-hydroxy-3-methylbutanoic acid; and
wherein the histidine analog is selected from the group consisting of β-(1,2,3-triazol-4-yl)-DL-alanine, and 1,2,4-triazole-3-alanine.

2. The modified polypeptide of claim 1, wherein the fatty acid is selected from the group consisting of palmitic acid, octanoic acid, hexanoic acid, docosahexaenoic acid, lauric acid, nonanoic acid, valeric acid, decanoic acid, oleic acid, arachidic acid, myristic acid, arachidonic acid, linoleic acid, stearic acid, decosanoic acid, tetracosanoic acid, sapienic acid, elaidic acid, vaccenic acid, eicosapentaenoic acid, and erucic acid.

3. The modified polypeptide of claim 1, wherein the fatty acid is attached to the N-terminus of the synthetic peptide.

4. The modified polypeptide of claim 1, wherein the fatty acid is attached to the C-terminus of the synthetic peptide.

5. The modified polypeptide of claim 3, wherein the synthetic polypeptide comprises a negatively charged C-terminal residue selected from the group consisting of aspartic acid, glutamic acid, methyl aspartic acid, methyl glutamic acid, 2-aminoadipic acid, 2-aminoheptanedioic acid, and iminodiacetic acid.

6. The modified polypeptide of claim 4, wherein the synthetic polypeptide comprises an N-terminal residue selected from the group consisting of glycine, lysine, arginine, citrulline, ornithine, and 2-amino-3-guanidinopropionic acid.

7. The modified polypeptide of claim 1, wherein the synthetic polypeptide comprises an amino acid sequence selected from the group consisting of:
(SEQ ID NO: 2)
Ala-Cys-Ala-His-Ala;
(SEQ ID NO: 3)
Ala-Ser-Ala-His-Ala;
(SEQ ID NO: 4)
Phe-Cys-Phe-His-Ala;
(SEQ ID NO: 5)
Phe-Ser-Phe-His-Ala;
(SEQ ID NO: 6)
Phe-His-Phe-Cys-Ala;
(SEQ ID NO: 7)
Phe-His-Phe-Ser-Ala;
(SEQ ID NO: 8)
Ala-Cys-Ala-His-Ala-Asp;
(SEQ ID NO: 9)
Ala-Ser-Ala-His-Ala-Asp;
(SEQ ID NO: 10)
Phe-Cys-Phe-His-Ala-Asp;
(SEQ ID NO: 11)
Phe-Ser-Phe-His-Ala-Asp;
(SEQ ID NO: 12)
Asp-Phe-His-Phe-Cys-Ala;
(SEQ ID NO: 13)
Asp-Phe-His-Phe-Ser-Ala;
(SEQ ID NO: 14)
Ala-Ala-Cys-Ala-His-Ala-Asp;
(SEQ ID NO: 15)
Ala-Ala-Ser-Ala-His-Ala-Asp;
(SEQ ID NO: 16)
Ala-Phe-Cys-Phe-His-Ala-Asp;
(SEQ ID NO: 17)
Ala-Phe-Ser-Phe-His-Ala-Asp;
(SEQ ID NO: 18)
Asp-Phe-His-Phe-Cys-Ala-Gly;
(SEQ ID NO: 19)
Asp-Phe-His-Phe-Ser-Ala-Gly;
(SEQ ID NO: 20)
Gly-Ala-Ala-Cys-Ala-His-Ala-Asp;
(SEQ ID NO: 21)
Gly-Ala-Ala-Ser-Ala-His-Ala-Asp;
(SEQ ID NO: 22)
Gly-Ala-Phe-Cys-Phe-His-Ala-Asp;
(SEQ ID NO: 23)
Gly-Ala-Phe-Ser-Phe-His-Ala-Asp;
(SEQ ID NO: 24)
Asp-Phe-His-Phe-Cys-Ala-Gly-Asp;
(SEQ ID NO: 25)
Asp-Phe-His-Phe-Ser-Ala-Gly-Asp;
(SEQ ID NO: 26)
Gly-Gly-Ala-Ala-Cys-Ala-His-Ala-Asp;
(SEQ ID NO: 27)
Arg-Gly-Ala-Ala-Cys-Ala-His-Ala-Asp;
(SEQ ID NO: 28)
Arg-Gly-Ala-Ala-Ser-Ala-His-Ala-Asp;
(SEQ ID NO: 29)
Arg-Gly-Ala-Phe-Cys-Phe-His-Ala-Asp;
(SEQ ID NO: 30)
Lys-Gly-Ala-Phe-Cys-Phe-His-Ala-Asp;
(SEQ ID NO: 31)
Arg-Gly-Ala-Phe-Ser-Phe-His-Ala-Asp;
(SEQ ID NO: 32)
Lys-Gly-Ala-Phe-Ser-Phe-His-Ala-Asp;
(SEQ ID NO: 33)
Arg-Asp-Phe-His-Phe-Cys-Ala-Gly-Asp;
and
(SEQ ID NO: 34)
Arg-Asp-Phe-His-Phe-Ser-Ala-Gly-Asp.

8. The modified polypeptide of claim 1, wherein the synthetic polypeptide comprises an amino acid sequence selected from the group consisting of:
(SEQ ID NO: 26)
Gly-Gly-Ala-Ala-Cys-Ala-His-Ala-Asp;
(SEQ ID NO: 27)
Arg-Gly-Ala-Ala-Cys-Ala-His-Ala-Asp;
(SEQ ID NO: 28)
Arg-Gly-Ala-Ala-Ser-Ala-His-Ala-Asp;
(SEQ ID NO: 29)
Arg-Gly-Ala-Phe-Cys-Phe-His-Ala-Asp;
(SEQ ID NO: 30)
Lys-Gly-Ala-Phe-Cys-Phe-His-Ala-Asp;
(SEQ ID NO: 31)
Arg-Gly-Ala-Phe-Ser-Phe-His-Ala-Asp;
(SEQ ID NO: 32)
Lys-Gly-Ala-Phe-Ser-Phe-His-Ala-Asp;
(SEQ ID NO: 33)
Arg-Asp-Phe-His-Phe-Cys-Ala-Gly-Asp;
and
(SEQ ID NO: 34)
Arg-Asp-Phe-His-Phe-Ser-Ala-Gly-Asp.

9. The modified polypeptide of claim 1, wherein the synthetic polypeptide is from 7 to 25 amino acids total in length.

10. The modified polypeptide of claim 1, wherein the synthetic polypeptide is from 8 to 20 amino acids total in length.

11. The modified polypeptide of claim 1, wherein the synthetic polypeptide is from 9 to 15 amino acids total in length.

12. A composition comprising one or more modified polypeptides of claim 1.

13. The composition of claim 12 further comprising a detergent.

14. The composition of claim 13, wherein the detergent is selected from the group consisting of polyoxyethylene octyl phenyl ether (Triton X-100), polyethylene glycol tert-octylphenyl ether (Triton X-114), polysorbate 20 (Tween-20), polysorbate 80 (Tween-80), nonylphenoxypolyethoxylethanol (NP-40), and octylphenoxypolyethoxyethanol (IGEPAL CA-630).

15. The composition of claim 13, wherein the one or more modified polypeptides and the detergent form a micelle.

16. A particle that is coated with one or more modified polypeptides of claim 1.

17. The particle of claim 16, wherein the particle is also coated with one or more amphiphilic polymers.

18. The particle of claim 17, wherein the one or more modified polypeptides are interspersed with the one or more amphiphilic polymers on the surface of the particle.

19. A composition comprising one or more particles of any of claims 16-18.

20. A cyclic polypeptide comprising the amino acid sequence of Gly-Glu-Ala-Glu-Ala-Glu-Gly-Pro-Gly-His-Ala-Glu-Ala-His-Gly-Pro (SEQ ID NO: 36).

21. The cyclic polypeptide of claim 20, wherein the four Glu residues and two His residues bind to two ferrous atoms.

22. A composition comprising the cyclic polypeptide of claim 20 or 21.

23. A composition comprising a DNA hairpin covalently coupled to four identical peptides comprising the amino acid sequence of Ala-Glu-Ala-His (SEQ ID NO: 37), wherein the DNA hairpin positions the four peptides in close proximity.

24. The composition of claim 22, wherein the Glu and His residues of the four peptides bind to two ferrous atoms.

25. A composition comprising a DNA origami structure covalently coupled to at least six peptides, wherein the six peptides are in close proximity and have either a Glu or His residue at its free terminus.

26. The composition of claim 24, wherein the Glu and His residues of the peptides bind to two ferrous atoms.

27.-32. (canceled)

33. A method of facilitating hydrolysis of a lipid comprising contacting the lipid with one or more modified polypeptides of claim 1.

34. (canceled)

35. The method of claim 33, wherein the one or more modified polypeptides are immobilized on the inner surface of channels in a cartridge or a flow-through device.

36. The method of claim 35, further comprising applying an external electric field to the cartridge or flow-through device.

37. (canceled)

38. The method of claim 36, wherein the external electrical field is applied in either one direction or in multiple directions.

39.-42. (canceled)


SEQUENCE LISTING (37 sequences)

SEQ ID NO: 1 - protein, 5 aa, Artificial Sequence
  synthetic polypeptide
XXXXX

SEQ ID NO: 2 - protein, 5 aa, Artificial Sequence
  synthetic polypeptide
ACAHA

SEQ ID NO: 3 - protein, 5 aa, Artificial Sequence
  synthetic polypeptide
ASAHA

SEQ ID NO: 4 - protein, 5 aa, Artificial Sequence
  synthetic polypeptide
FCFHA

SEQ ID NO: 5 - protein, 5 aa, Artificial Sequence
  synthetic polypeptide
FSFHA

SEQ ID NO: 6 - protein, 5 aa, Artificial Sequence
  synthetic polypeptide
FHFCA

SEQ ID NO: 7 - protein, 5 aa, Artificial Sequence
  synthetic polypeptide
FHFSA

SEQ ID NO: 8 - protein, 6 aa, Artificial Sequence
  synthetic polypeptide
ACAHAD

SEQ ID NO: 9 - protein, 6 aa, Artificial Sequence
  synthetic polypeptide
ASAHAD

SEQ ID NO: 10 - protein, 6 aa, Artificial Sequence
  synthetic polypeptide
FCFHAD

SEQ ID NO: 11 - protein, 6 aa, Artificial Sequence
  synthetic polypeptide
FSFHAD

SEQ ID NO: 12 - protein, 6 aa, Artificial Sequence
  synthetic polypeptide
DFHFCA

SEQ ID NO: 13 - protein, 6 aa, Artificial Sequence
  synthetic polypeptide
DFHFSA

SEQ ID NO: 14 - protein, 7 aa, Artificial Sequence
  synthetic polypeptide
AACAHAD

SEQ ID NO: 15 - protein, 7 aa, Artificial Sequence
  synthetic polypeptide
AASAHAD

SEQ ID NO: 16 - protein, 7 aa, Artificial Sequence
  synthetic polypeptide
AFCFHAD

SEQ ID NO: 17 - protein, 7 aa, Artificial Sequence
  synthetic polypeptide
AFSFHAD

SEQ ID NO: 18 - protein, 7 aa, Artificial Sequence
  synthetic polypeptide
DFHFCAG

SEQ ID NO: 19 - protein, 7 aa, Artificial Sequence
  synthetic polypeptide
DFHFSAG

SEQ ID NO: 20 - protein, 8 aa, Artificial Sequence
  synthetic polypeptide
GAACAHAD

SEQ ID NO: 21 - protein, 8 aa, Artificial Sequence
  synthetic polypeptide
GAASAHAD

SEQ ID NO: 22 - protein, 8 aa, Artificial Sequence
  synthetic polypeptide
GAFCFHAD

SEQ ID NO: 23 - protein, 8 aa, Artificial Sequence
  synthetic polypeptide
GAFSFHAD

SEQ ID NO: 24 - protein, 8 aa, Artificial Sequence
  synthetic polypeptide
DFHFCAGD

SEQ ID NO: 25 - protein, 8 aa, Artificial Sequence
  synthetic polypeptide
DFHFSAGD

SEQ ID NO: 26 - protein, 9 aa, Artificial Sequence
  synthetic polypeptide
GGAACAHAD

SEQ ID NO: 27 - protein, 9 aa, Artificial Sequence
  synthetic polypeptide
RGAACAHAD

SEQ ID NO: 28 - protein, 9 aa, Artificial Sequence
  synthetic polypeptide
RGAASAHAD

SEQ ID NO: 29 - protein, 9 aa, Artificial Sequence
  synthetic polypeptide
RGAFCFHAD

SEQ ID NO: 30 - protein, 9 aa, Artificial Sequence
  synthetic polypeptide
KGAFCFHAD

SEQ ID NO: 31 - protein, 9 aa, Artificial Sequence
  synthetic polypeptide
RGAFSFHAD

SEQ ID NO: 32 - protein, 9 aa, Artificial Sequence
  synthetic polypeptide
KGAFSFHAD

SEQ ID NO: 33 - protein, 9 aa, Artificial Sequence
  synthetic polypeptide
RDFHFCAGD

SEQ ID NO: 34 - protein, 9 aa, Artificial Sequence
  synthetic polypeptide
RDFHFSAGD

SEQ ID NO: 35 - protein, 11 aa, Artificial Sequence
  synthetic polypeptide
HGGPGGHGCGD

SEQ ID NO: 36 - protein, 16 aa, Artificial Sequence
  synthetic polypeptide
GEAEAEGPGHAEAHGP

SEQ ID NO: 37 - protein, 4 aa, Artificial Sequence
  synthetic polypeptide
AEAH